(19) United States
(12) Patent Application Publication
(10) Pub. No.: US 2022/0064271 A1
(43) Pub. Date: Mar. 3, 2022

(54) METHODS FOR TREATING TAUOPATHIES

(71) Applicant: Washington University, St. Louis, MO (US)

(72) Inventors: Randall Bateman, St. Louis, MO (US); David Holtzman, St. Louis, MO (US); Kanta Horie, St. Louis, MO (US); Nicolas Barthelemy, St. Louis, MO (US); Chihiro Sato, St. Louis, MO (US); Hong Jiang, St. Louis, MO (US)

(21) Appl. No.: 17/464,190

(22) Filed: Sep. 1, 2021

Related U.S. Application Data
(60) Provisional application No. 63/073,187, filed on Sep. 1, 2020.

(51) Int. Cl. C07K 16/18; G01N 33/68

(52) U.S. Cl. CPC C07K 16/18; C07K 2317/34; G01N 33/6896

(57) ABSTRACT

This invention relates to epitope binding agents that specifically bind to tau and methods of use thereof. The disclosure provides immunoassays, kits, and pharmaceutical compositions which include an epitope binding agent that specifically bind to tau. The disclosure provides methods of reducing the spread of tau aggregates and methods of reducing a tauopathy-related pathology.

### CROSS REFERENCE TO RELATED APPLICATIONS

This application claims the benefit of U.S. Provisional Application No. 63/073,187, filed Sep. 1, 2020 the disclosure of which is herein incorporated by reference in its entirety.

### REFERENCE TO SEQUENCE LISTING

This application contains a Sequence Listing that has been submitted in ASCII format via EFS-Web and is hereby incorporated by reference in its entirety. The ASCII copy, created on Aug. 31, 2021, is named 698703_ST25.txt, and is 8,345 bytes in size.

### FIELD

This invention relates to epitope binding agents that specifically bind to tau and methods of use thereof.

### BACKGROUND

Accumulation of tau protein as insoluble aggregates in the brain is one of the hallmarks of Alzheimer's disease and other neurodegenerative diseases called tauopathies. Tau pathology appears to propagate across brain regions and spread by the transmission of specific pathological tau species from cell to cell in a prion-like manner although the nature of these species (i.e., monomeric, oligomeric, and fibril species) and the spreading process are uncertain (Frost et al., 2009; Goedert et al., 2010, 2017; Sanders et al., 2014; Wu et al., 2016; Mirbaha et al., 2018; Lasagna-Reeves et al., 2012). Tau has six different isoforms of the full-length protein. In addition, tau has more than one hundred potential post-translational modification sites, including phosphorylation, in addition to multiple truncation sites (Meredith et al., 2013; Sato et al., 2018; Barthélemy et al., 2019; Cicognola et al., 2019; Blennow et al., 2020). Thus, identifying specific pathological tau species involved in tau spread is challenging and, consequently, only symptomatic treatments are currently available for tauopathies with mild or no efficacy.

Accordingly, there remains a need in the art for new compounds and compositions useful in the treatment of tauopathies.

### BRIEF DESCRIPTION OF THE FIGURES

The application file contains at least one photograph executed in color. Copies of this patent application publication with color photographs will be provided by the Office upon request and payment of the necessary fee.

FIG. 1 is a schematic of the longest human tau isoform (2N4R). The N-terminus (N term), mid domain, MTBR, and C-terminus (C term) are identified for this isoform and will vary in a predictable way for other tau isoforms (e.g., 2N3R, 1NR4, 1N3R, 0N4R, and 0N3R).

FIG. 2 is a schematic illustrating several methods to process CSF or blood samples prior to quantitative measurement of tau or other CNS proteins (e.g., by an immune-assay, by mass spectrometry, etc.). The method detailed within the blue box (right) is one method, referred to as “CX method” when the starting material is a blood or CSF sample. The method detailed within the red box (left) is a second method, referred to as “IP method.” A blood sample may be used rather than a CSF sample. The combination of the red box (left) and the blue box (right) is another method, referred to as “PostIP-CX method”.

FIG. 3A is a schematic of tryptic peptides from tau (grey bars) that were quantified in Example 1, and further discussed in FIG. 3B and FIG. 3C.

FIG. 3B and FIG. 3C are graphs showing brain MTBR tau species comprising MTBR tau-243, 299 and 354 are enriched in aggregated Alzheimer's disease brain insoluble extracts compared to control brain extracts, confirming that MTBR tau is specifically deposited in Alzheimer's disease brain. The graphs show the enrichment profile of tau peptides from (FIG. 3B) control and Alzheimer's disease brains (n=2 with six-eight brain regions samples/group in discovery cohort) and (FIG. 3C) from control (amyloid-negative, n=8), very mild to moderate Alzheimer's disease (AD) (amyloid-positive, CDR=0.5-2, n=5), and severe AD brains (amyloid-positive, CDR=3, n=7) (total n=20 in validation cohort). The relative abundance of tau peptides was quantified relative to the mid-domain (residue 181-190) peptide for internal normalization. The species containing the upstream region of microtubule binding region (MTBR) domain (residue 243-254, MTBR tau-243) and repeat region 2 (R2) to R3 and R4 (residues 299-317, MTBR tau-299 and 354-369, MTBR tau-354, respectively) were highly enriched in the insoluble fraction from Alzheimer's disease brains compared to controls and were specifically enriched by clinical stage of disease progression as measured by the CDR. MTBR tau-299 and MTBR tau-354 are located inside the filament core, whereas MTBR tau-243 is located outside the core of Alzheimer's disease aggregates (Fitzpatrick et al., 2017). Of note, residue 195-209 was decreased in Alzheimer's disease brains, potentially due to a high degree of phosphorylation. Data are represented as box-and-whisker plots with Tukey method describing median, interquartile interval, minimum, maximum, and individual points for outliers. Significance in statistical test: ****p<0.001, ***p<0.001, **p<0.01, *p<0.05.

FIG. 4A is a schematic of tryptic peptides from tau (grey bars) that were quantified in Example 1, and further discussed in FIG. 4B, as well as the general binding site of the antibodies HJ8.5 and Tau1.

FIG. 4B is a graph showing the tau profile in control human CSF. Tau peptides in control human CSF from a cross-sectional cohort of amyloid-negative and CDR=0 patients (n=30) were quantified by Tau1/HJ8.5 immunoprecipitation focusing on N-terminal to mid-domain tau. To quantify the species containing the microtubule binding region (MTBR) and C-terminal region, post-immunoprecipitated CSF samples were chemically extracted and analyzed sequentially. Using the Tau1/HJ8.5 immunoprecipitation method (blue circle), peptide recovery dramatically decreased after reside 222; therefore, only N-terminal to mid-domain tau (residues 6-23 to 243-254) peptides were quantified by this method (Sato et al., 2018). In contrast, the chemical extraction method of post-immunoprecipitated CSF (red square) enabled quantification of whole regions of tau including the MTBR to C-terminal regions at concentrations between 0.4-7 ng/mL. Data are represented as means.

FIG. 5A, FIG. 5B, and FIG. 5C are graphs showing the amount of mid-domain-independent MTBR tau-243 (FIG. 5A), mid-domain-independent MTBR tau-299 (FIG. 5B), and mid-domain-independent MTBR tau-354 (FIG. 5C) in PostIP-CX CSF from the cross-sectional cohort. Mid-domain-independent MTBR tau-243, mid-domain-independent MTBR tau-299 and mid-domain-independent MTBR tau-354 show different profiles to amyloid plaques and clinical dementia stage. Amyloid-negative CDR=0 (control, n=30), amyloid-positive CDR=0 (preclinical AD, n=18), amyloid-positive CDR=0.5 (very mild AD, n=28), amyloid-positive CDR≥1 (mild-moderate AD, n=12), and amyloid-negative CDR≥0.5 (non-AD cognitive impairment, n=12). Mid-domain-independent MTBR tau-243 showed a continuous increase with Alzheimer's disease progression through all clinical stages. Mid-domain-independent MTBR tau-299 and mid-domain-independent MTBR tau-354 concentrations similarly increased until the very mild Alzheimer's disease stage (amyloid-positive and CDR=0.5), but then either saturated (MTBR tau-299) or decreased (MTBR tau-354) at CDR≥1. The p-values in red or blue fonts indicate a significant increase or decrease, respectively. Data are represented as the individual results (plots) and the mean (bar). Significance in statistical test: ****p<0.001, ***p<0.001, **p<0.01, *p<0.05. NS=not significant.

FIG. 6A, FIG. 6B, and FIG. 6C are graphs showing longitudinal rates of changes (ng/mL/year) in (FIG. 6A) mid-domain-independent MTBR tau-243, (FIG. 6B) mid-domain-independent MTBR tau-299, and (FIG. 6C) mid-domain-independent MTBR tau-354 in CSF in amyloid-negative (−) or positive (+) patients are shown (total n=28 from longitudinal cohort). Amyloid-positive groups are further divided to various CDR changes including CDR=0-0 (n=7), CDR=0-0.5 (n=2), CDR=0-1 (n=1), CDR=0.5-0.5 (n=2), CDR=0.5-1 (n=1), and CDR=1-2 (n=1, participant A). While the participants in the amyloid-negative group did not show significant longitudinal changes (as mean-values are close to zero), most participants in the amyloid-positive group showed increases in MTBR tau concentrations longitudinally. Notably, participant A with the greatest cognitive change after Alzheimer's disease clinical onset (CDR=1 to 2) demonstrated only CSF MTBR tau-243 increased with mild AD (CDR=1) to moderate AD (CDR=2) progression, while MTBR tau-299 and 354 decreased. These data show CSF mid-domain-independent MTBR tau species longitudinally increase with advancing Alzheimer's disease clinical stages.

FIG. 7A, FIG. 7B, and FIG. 7C are graphs showing (x-axis) tau PET (AV-1451) SUVR and (y-axis) mid-domain-independent (FIG. 7A) MTBR tau-243, (FIG. 7B) MTBR tau-299, and (FIG. 7C) MTBR tau-354 concentrations in PostIP-CX CSF (control n=15 and Alzheimer's disease (AD) n=20 from tau PET cohort). Open circle: control, filled squares: AD. Mid-domain-independent MTBR tau-243 showed the most significant correlation with tau PET SUVR (r=0.7588, p<0.0001). These data show CSF mid-domain-independent MTBR tau species comprising SEQ ID NO: 3 (LQTAPVPMPDLK) is highly correlated with tau PET SUVR measure of tau tangles, while other mid-domain-independent MTBR tau regions have lower correlations with tau tangles.

FIG. 8A and FIG. 8B graphically depict that brain MTBR tau-243, MTBR tau-299, and MTBR tau-354 are not enriched in Alzheimer's disease brain soluble extracts compared to control brain extracts. FIG. 8A shows the enrichment profile of tau peptides from control and Alzheimer's disease brains (n=2 with eight-ten brain regions samples/group in discovery cohort) and FIG. 8B shows the enrichment profile of tau peptides from control (amyloid-negative, n=8), very mild to moderate Alzheimer's disease (AD) (amyloid-positive, CDR=0.5-2, n=5), and severe AD brains (amyloid-positive, CDR=3, n=7) in the validation cohort (total n=20). Relative peptide abundance of tau peptides was quantified relative to mid-domain (residue 181-190) peptide for internal normalization. No changes were observed for tau species containing microtubule binding region (MTBR) domain in soluble tau species, in contrast to the insoluble MTBR tau species which were increased in Alzheimer's disease brains (FIG. 3). Data are represented as box-and-whisker plots with Tukey method describing median, interquartile interval, minimum, maximum, and individual points for outlier. Significance in statistical test: **p<0.01, *p<0.05.

FIG. 9 is a graph showing that MTBR tau-354 correlates with tau368 in brain insoluble extracts, suggesting each species is not differentiated by progression of tau pathology. Mass spectrometry analysis of MTBR tau-354 and tau368 species in brain insoluble extracts from control and Alzheimer's disease patients was conducted using discovery cohort samples (total 23 brain samples, six brain regions from Alzheimer's disease #1 participant, eight brain regions from Alzheimer's disease #2 participant, five brain regions from Control #1 participant, four brain regions from Control #2 participant). MTBR tau-354 (residue 354-369) and its truncated form, tau368 (residue 354-368), exhibited a tight correlation in brain insoluble extracts (Spearman r=0.9783).

FIG. 10A, FIG. 10B, FIG. 10C, FIG. 10D, FIG. 10E and FIG. 10F are graphs showing the quantification of tryptic peptides of MTBR tau in a human CSF sample after processing by the PostIP-CX method followed by mass spectrometry (MS) analysis. Extracted MS chromatograms of mid-domain-independent MTBR tau-243 (FIG. 10A, FIG. 10D), mid-domain-independent MTBR tau-299 (FIG. 10B, FIG. 10E), and mid-domain-independent MTBR tau-354 (FIG. 10C, FIG. 10F) are shown. The human CSF was obtained from an amyloid positive and CDR=0.5 very mild Alzheimer's disease participant from the cross-sectional cohort. FIG. 10A, FIG. 10B, and FIG. 10C show the peaks from endogenous tryptic peptides, while FIG. 10D, FIG. 10E, and FIG. 10F show the peaks from an internal standard (¹⁵N-labeled tau). X-axis and Y-axis indicate the retention time and MS intensity of each peak, respectively.

FIG. 11A, FIG. 11B, FIG. 11C, FIG. 11D, FIG. 11E, FIG. 11F, FIG. 11G, FIG. 11H, FIG. 11I, FIG. 11J, and FIG. 11K are graphs showing the concentration of tryptic peptides of N-terminal tau and mid-domain tau in human CSF after sample processing by the IP method followed by MS analysis. N-terminal and mid-domain CSF tau species distinguish very early dementia from normal, but do not correlate with dementia stage. Tau species, residues (FIG. 11A) 6-23, (FIG. 11B) 25-44, (FIG. 11C) 45-67, (FIG. 11D) 68-87, (FIG. 11E) 88-126, (FIG. 11F) 151-155, (FIG. 11G) 181-190, (FIG. 11H) 195-209, (FIG. 11I) 212-221, (FIG. 11J) 226-230, and (FIG. 11K) 243-254 concentrations in CSF from groups within the cross-sectional cohort (residue numbering based on tau-441). Amyloid-negative CDR=0 (control, n=29), amyloid-positive CDR=0 (preclinical AD, n=18), amyloid-positive CDR=0.5 (very mild AD, n=27), amyloid-positive CDR≥1 (mild-moderate AD, n=12), and amyloid-negative CDR≥0.5 (non-AD clinical impairment, n=12). These tau species were isolated using the Tau1/HJ8.5 immunoprecipitation method (IP method). The p-values in red font indicate a significance. Data are represented as the individual results (plots) and the mean (bar). Significance in statistical test: ****p<0.001, ***p<0.001, **p<0.01, *p<0.05. NS=not significant.

FIG. 12A, FIG. 12B, FIG. 12C, FIG. 12D, FIG. 12E, FIG. 12F, FIG. 12G, and FIG. 12H are graphs showing the concentration of tryptic peptides of MTBR tau in human CSF after sample processing by the PostIP-CX method followed by MS analysis. Unique Alzheimer's disease amyloid and clinical staging patterns are specific to mid-domain-independent MTBR tau species in CSF. Only mid-domain-independent MTBR tau-243 distinguishes more advanced clinical stages. Tau species, residues (FIG. 12A) 243-254 (MTBR tau-243), (FIG. 12B) 260-267, (FIG. 12C) 275-280, (FIG. 12D) 282-290, (FIG. 12E) 299-317 (MTBR tau-299), (FIG. 12F) 354-369 (MTBR tau-354), (FIG. 12G) 386-395, and (FIG. 12H) 396-406 concentrations in CSF obtained using chemical extraction from post-immunoprecipitated samples from participants within the cross-sectional cohort (residue numbering based on tau-441). Amyloid-negative CDR=0 (control, n=30), amyloid-positive CDR=0 (preclinical AD, n=18), amyloid-positive CDR=0.5 (very mild AD, n=28), amyloid-positive CDR1 (mild-moderate AD, n=12), and amyloid-negative CDR0.5 (non-AD clinical impairment, n=12). The p-values in red or blue fonts indicate a significant increase or decrease, respectively. Data are represented as the individual results (plots) and the mean (bar). Significance in statistical test: ****p<0.001, ***p<0.001, **p<0.01, *p<0.05. NS=not significant.

FIG. 13A, FIG. 13B, FIG. 13C, FIG. 13D, FIG. 13E, FIG. 13F, FIG. 13G, FIG. 13H, FIG. 13I, and FIG. 13J are graphs showing the concentration of tryptic peptides of N-terminal tau and mid-domain tau in human CSF after sample processing by the PostIP-CX method followed by MS analysis. N-terminal and mid-domain CSF tau species do not correlate with dementia stage regardless of the purification method (see also FIG. 26). Tau species, residues (FIG. 13A) 6-23, (FIG. 13B) 25-44, (FIG. 13C) 45-67, (FIG. 13D) 68-87, (FIG. 13E) 88-126, (FIG. 13F) 151-155, (FIG. 13G) 181-190, (FIG. 13H) 195-209, (FIG. 13I) 212-221, and (FIG. 13J) 226-230, concentrations in CSF from groups within the cross-sectional cohort. Amyloid-negative CDR=0 (control, n=29), amyloid-positive CDR=0 (preclinical AD, n=18), amyloid-positive CDR=0.5 (very mild AD, n=27), amyloid-positive CDR1 (mild-moderate AD, n=12), and amyloid-negative CDR0.5 (non-AD clinical impairment, n=12). These tau species were isolated using the Tau1/HJ8.5 immunoprecipitation method followed by chemical extraction of the post-immunoprecipitated CSF (PostIP-CX). The sums of concentrations from the immunoprecipitation method and chemical extraction method for post-immunoprecipitated CSF are shown for N-terminal to mid domain tau species (as total concentrations). The p-values in red font indicate statistical significance. Data are represented as the individual results (plots) and the mean (bar). Significance in statistical test: ****p<0.001, ***p<0.001, **p<0.01, *p<0.05. NS=not significant.

FIG. 13K is a graph showing the total concentration of tau species containing residues 243-254 (sum concentration from the IP method and the PostIP-CX method).

FIG. 14 graphically shows that mid-domain-independent MTBR tau-354 correlates with mid-domain-independent tau368 in CSF. CSF was processed by the PostIP-CX method. Mid-domain-independent MTBR tau-354 (residue 354-369) and its truncated form, tau368 (residue 354-368), display a tight correlation (Spearman r=0.8382). Results were obtained from cross-sectional cohort samples (n=100 including all clinical stages).

FIG. 15A, FIG. 15B, and FIG. 15C are graphs showing that CSF mid-domain-independent MTBR tau-243 highly correlates with Clinical dementia rating—sum of boxes (CDR-SB), while mid-domain-independent MTBR tau-299 and mid-domain-independent MTBR tau-354 do not. CDR-SB correlations with (FIG. 15A) mid-domain-independent MTBR tau-243, (FIG. 15B) mid-domain-independent MTBR tau-299, and (FIG. 30C) mid-domain-independent MTBR tau-354 concentrations in CSF. Mid-domain-independent MTBR tau-243 in CSF from amyloid positive groups showed a high correlation with CDR-SB (r=0.5562, p<0.0001). Results were obtained from cross-sectional cohort samples (amyloid-negative n=42 and amyloid-positive n=58).

FIG. 16A, FIG. 16B, and FIG. 16C are graphs showing that CSF mid-domain-independent MTBR tau-243 highly correlates with mini-mental state exam (MMSE) more than mid-domain-independent MTBR tau-299 and mid-domain-independent MTBR tau-354. MMSE) correlations with (FIG. 16A) mid-domain-independent MTBR tau-243, (FIG. 16B) mid-domain-independent MTBR tau-299, and (FIG. 16C) mid-domain-independent MTBR tau-354 concentrations in CSF. Mid-domain-independent MTBR tau-243 in CSF from amyloid positive groups showed a high correlation with MMSE (r=0.5433, p<0.0001). Results were obtained from cross-sectional cohort samples (amyloid-negative n=42 and amyloid-positive n=58).

FIG. 17A, FIG. 17B, and FIG. 17C are graphs showing CSF mid-domain-independent MTBR tau-243, mid-domain-independent MTBR tau-299, mid-domain-independent MTBR tau-354 longitudinally increase with advancing Alzheimer's disease clinical stages. Longitudinal changes in mid-domain-independent (FIG. 17A) MTBR tau-243, (FIG. 17B) MTBR tau-299, and (FIG. 17C) MTBR tau-354 tau concentrations in CSF in amyloid negative (−) or positive (+) patients are shown. Black circle: CDR=0, Blue triangle: CDR=0.5, Red square: CDR=1, Purple reverse-triangle: CDR=2. The participants with stable (or decreasing) CDR trajectory are represented by a dotted line. The participants with increasing CDR between the 1st and 2nd visits are represented by a solid line. The bolded red line in the amyloid-positive group shows the longitudinal trajectory of a specific participant (participant A) with the greatest cognitive change after Alzheimer's disease onset (CDR=1 to 2), indicating CSF MTBR tau-243 increased even in mild AD (CDR=1) to moderate AD (CDR=2). Statistical significance was evaluated by paired t-test for 1^{st} and 2^{nd} visits (amyloid-negative n=14 and amyloid-positive n=14). **p<0.01. NS=not significant.

FIG. 18 is an illustration of a theoretical model showing how accessibility of different regions of MTBR tau to cleavage may vary during Alzheimer's disease (AD) progression, and why MTBR tau species comprising the amino acid sequence of SEQ ID NO: 3 (LQTAPVPMPDLK) is a good surrogate for tau-pathology all across AD stages. In preclinical AD stages, brain tau aggregates are immature, allowing proteases greater access. MTBR tau species comprising MTBR tau-243, MTBR tau-299, and MTBR tau-354 are secreted into CSF. However, as tau aggregates mature with disease progression and form an increasingly rigid core, protease access to MTBR tau-354 and then MTBR tau-299 decreases, and MTBR-species comprising MTBR tau-354 and then MTBR tau-299 stabilize in the CSF. MTBR tau-243, however, remains exposed, enabling protease digestion and release into CSF at all disease stages. The imbalance for these three species in CSF is observed as a reflection of brain tau aggregate formation. Note: in this illustration, differences in size between MTBR tau species is not depicted.

### DETAILED DESCRIPTION

The present disclosure provides epitope binding agents that specifically bind to certain disease-specific MTBR tau species, including mid-domain-independent MTBR tau. MTBR tau exists as a plurality of peptides in blood and CSF. The term “mid-domain-independent MTBR tau” refers to a plurality of MTBR tau species that lack all or substantially all of the mid-domain region of tau, and therefore also the N-terminus region. These mid-domain-independent MTBR tau species remain after tau species comprising mid-domain tau have been depleted, partially or completely, from a biological sample, preferably from a blood or CSF sample. PCT International Application No. PCT/US2020/046224, the disclosures of which are incorporated herein by reference, provides improved methods to quantify MTBR tau, in particular mid-domain-independent MTBR tau species. As detailed in Example 1, these tau species are particularly suited for measuring clinical signs and symptoms of tauopathies, diagnose tauopathies, and direct treatment of tauopathies.

In addition to providing epitope binding agents that specifically bind to certain disease-specific MTBR tau species, the present disclosure also provides therapeutic and diagnostic uses of these agents.

These and other aspects and iterations of the invention are described more thoroughly below.

### I. Definitions

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

The term “Aβ” refers to peptides derived from a region in the carboxy terminus of a larger protein called amyloid precursor protein (APP). The gene encoding APP is located on chromosome 21. There are many forms of Aβ that may have toxic effects: Aβ peptides are typically 37-43 amino acid sequences long, though they can have truncations and modifications changing their overall size. They can be found in soluble and insoluble compartments, in monomeric, oligomeric and aggregated forms, intracellularly or extracellularly, and may be complexed with other proteins or molecules. The adverse or toxic effects of Aβ may be attributable to any or all of the above noted forms, as well as to others not described specifically. For example, two such Aβ isoforms include Aβ40 and Aβ42; with the Aβ42 isoform being particularly fibrillogenic or insoluble and associated with disease states. The term “Aβ” typically refers to a plurality of Aβ species without discrimination among individual Aβ species. Specific Aβ species are identified by the size of the peptide, e.g., Aβ42, Aβ40, Aβ38 etc.

As used herein, the term “Aβ42/Aβ40 value” means the ratio of the amount of Aβ42 in a sample obtained from a subject compared to the amount of Aβ40 in the same sample.

“Aβ amyloidosis” is defined as clinically abnormal Aβ deposition in the brain. A subject that is determined to have Aβ amyloidosis is referred to herein as “amyloid positive,” while a subject that is determined to not have Aβ amyloidosis is referred to herein as “amyloid negative.” There are accepted indicators of Aβ amyloidosis in the art. At the time of this disclosure, Aβ amyloidosis is directly measured by amyloid imaging (e.g., PiB PET, fluorbetapir, or other imaging methods known in the art) or indirectly measured by decreased cerebrospinal fluid (CSF) Aβ42 or a decreased CSF Aβ42/40 ratio. [11C]PIB-PET imaging with mean cortical binding potential (MCBP) score>0.18 is an indicator of Aβ amyloidosis, as is cerebral spinal fluid (CSF) Aβ42 concentration of about 1 ng/ml measured by immunoprecipitation and mass spectrometry (IP/MS)). Alternatively, a cut-off ratio for CSF Aβ42/40 that maximizes the accuracy in predicting amyloid-positivity as determined by PIB-PET can be used. Values such as these, or others known in the art and/or used in the examples, may be used alone or in combination to clinically confirm Aβ amyloidosis. See, for example, Klunk W E et al. Ann Neurol 55(3) 2004, Fagan A M et al. Ann Neurol, 2006, 59(3), Patterson et. al, Annals of Neurology, 2015, 78(3): 439-453, or Johnson et al., J. Nuc. Med., 2013, 54(7): 1011-1013, each hereby incorporated by reference in its entirety. Subjects with Aβ amyloidosis may or may not be symptomatic, and symptomatic subjects may or may not satisfy the clinical criteria for a disease associated with Aβ amyloidosis. Non-limiting examples of symptoms associated with Aβ amyloidosis may include impaired cognitive function, altered behavior, abnormal language function, emotional dysregulation, seizures, dementia, and impaired nervous system structure or function. Diseases associated with Aβ amyloidosis include, but are not limited to, Alzheimer's Disease (AD), cerebral amyloid angiopathy (CAA), Lewy body dementia, and inclusion body myositis. Subjects with Aβ amyloidosis are at an increased risk of developing a disease associated with Aβ amyloidosis.

A “clinical sign of Aβ amyloidosis” refers to a measure of Aβ deposition known in the art. Clinical signs of Aβ amyloidosis may include, but are not limited to, Aβ deposition identified by amyloid imaging (e.g. PiB PET, fluorbetapir, or other imaging methods known in the art) or by decreased cerebrospinal fluid (CSF) Aβ42 or Aβ42/40 ratio. See, for example, Klunk W E et al. Ann Neurol 55(3) 2004, and Fagan A M et al. Ann Neurol 59(3) 2006, each hereby incorporated by reference in its entirety. Clinical signs of Aβ amyloidosis may also include measurements of the metabolism of Aβ, in particular measurements of Aβ42 metabolism alone or in comparison to measurements of the metabolism of other Aβ variants (e.g. Aβ37, Aβ38, Aβ39, Aβ40, and/or total Aβ), as described in U.S. patent application Ser. No. 14/366,831, Ser. No. 14/523,148 and Ser. No. 14/747,453, each hereby incorporated by reference in its entirety. Additional methods are described in Albert et al. Alzheimer's & Dementia 2007 Vol. 7, pp. 170-179; McKhann et al., Alzheimer's & Dementia 2007 Vol. 7, pp. 263-269; and Sperling et al. Alzheimer's & Dementia 2007 Vol. 7, pp. 280-292, each hereby incorporated by reference in its entirety. Importantly, a subject with clinical signs of Aβ amyloidosis may or may not have symptoms associated with Aβ deposition. Yet subjects with clinical signs of Aβ amyloidosis are at an increased risk of developing a disease associated with Aβ amyloidosis.

A “candidate for amyloid imaging” refers to a subject that has been identified by a clinician as an individual for whom amyloid imaging may be clinically warranted. As a non-limiting example, a candidate for amyloid imaging may be a subject with one or more clinical signs of Aβ amyloidosis, one or more Aβ plaque associated symptoms, one or more CAA associated symptoms, or combinations thereof. A clinician may recommend amyloid imaging for such a subject to direct his or her clinical care. As another non-limiting example, a candidate for amyloid imaging may be a potential participant in a clinical trial for a disease associated with Aβ amyloidosis (either a control subject or a test subject).

As used herein, the term “subject” refers to a mammal, preferably a human. The mammals include, but are not limited to, humans, primates, livestock, rodents, and pets. A subject may be waiting for medical care or treatment, may be under medical care or treatment, or may have received medical care or treatment.

As used herein, the term “control population,” “normal population” or a sample from a “healthy” subject refers to a subject, or group of subjects, who are clinically determined to not have a tauopathy or Aβ amyloidosis, or a clinical disease associated with Aβ amyloidosis (including but not limited to Alzheimer's disease), based on qualitative or quantitative test results.

As used herein, the term “blood sample” refers to a biological sample derived from blood, preferably peripheral (or circulating) blood. The blood sample can be whole blood, plasma or serum, although plasma is typically preferred.

The term “isoform”, as used herein, refers to any of several different forms of the same protein variants, arising due to alternative splicing of mRNA encoding the protein, post-translational modification of the protein, proteolytic processing of the protein, genetic variations and somatic recombination. The terms “isoform” and “variant” are used interchangeably.

The term “tau” refers to a plurality of isoforms encoded by the gene MAPT (or homolog thereof), as well as species thereof that are C-terminally truncated in vivo, N-terminally truncated in vivo, post-translationally modified in vivo, or any combination thereof. As used herein, the terms “tau” and “tau protein” and “tau species” may be used interchangeably. In many animals, including but not limited to humans, non-human primates, rodents, fish, cattle, frogs, goats, and chicken, tau is encoded by the gene MAPT. In animals where the gene is not identified as MAPT, a homolog may be identified by methods well known in the art.

In humans, there are six isoforms of tau that are generated by alternative splicing of exons 2, 3, and 10 of MAPT. These isoforms range in length from 352 to 441 amino acids. Exons 2 and 3 encode 29-amino acid inserts each in the N-terminus (called N), and full-length human tau isoforms may have both inserts (2N), one insert (1N), or no inserts (0N). All full-length human tau isoforms also have three repeats of the microtubule binding domain (called R). Inclusion of exon 10 at the C-terminus leads to inclusion of a fourth microtubule binding domain encoded by exon 10. Hence, full-length human tau isoforms may be comprised of four repeats of the microtubule binding domain (exon 10 included: R1, R2, R3, and R4) or three repeats of the microtubule binding domain (exon 10 excluded: R1, R3, and R4). Human tau may or may not be post-translationally modified. For example, it is known in the art that tau may be phosphorylated, ubiquinated, glycosylated, and glycated. Human tau also may or may not be proteolytically processed in vivo at the C-terminus, at the N-terminus, or at the C-terminus and the N-terminus. Accordingly, the term “human tau” encompasses the 2N3R, 2N4R, 1N3R, 1N4R, 0N3R, and 0N4R isoforms, as well as species thereof that are C-terminally truncated in vivo, N-terminally truncated in vivo, post-translationally modified in vivo, or any combination thereof. Alternative splicing of the gene encoding tau similarly occurs in other animals.

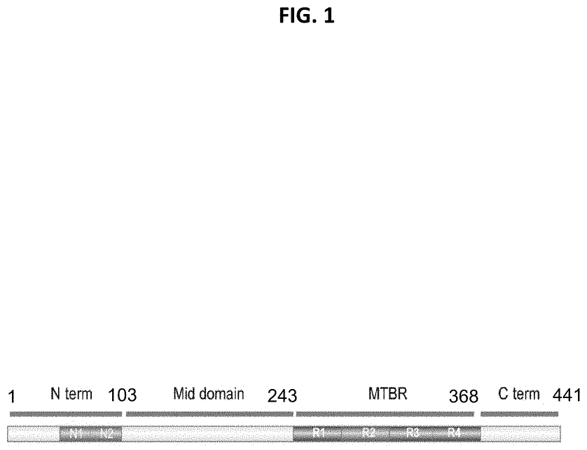

The term “tau-441,” as used herein, refers to the longest human tau isoform (2N4R), which is 441 amino acids in length. The amino acid sequence of tau-441 is provided as SEQ ID NO: 1 (MAEPRQEFEVMEDHAGTYGLGDRKDQGGYTMHQDQEGDTDAGLKESPLQTPTEDG SEEPGSETSDAKSTPTAEDVTAPLVDEGAPGKQAAAQPHTEIPEGTTAEEAGIGDTPSL EDEAAGHVTQARMVSKSKDGTGSDDKKAKGADGKTKIATPRGAAPPGQKGQANATRI PAKTPPAPKTPPSSGEPPKSGDRSGYSSPGSPGTPGSRSRTPSLPTPPTREPKKVAVV RTPPKSPSSAKSRLQTAPVPMPDLKNVKSKIGSTENLKHQPGGGKVQIINKKLDLSNVQ SKCGSKDNIKHVPGGGSVQIVYKPVDLSKVTSKCGSLGNIHHKPGGGQVEVKSEKLDF KDRVQSKIGSLDNITHVPGGGNKKIETHKLTFRENAKAKTDHGAEIVYKSPVVSGDTSP RHLSNVSSTGSIDMVDSPQLATLADEVSASLAKQGL). The N-terminus (N term), mid-domain, MTBR, and C-terminus (C term) are identified in FIG. 1 for this isoform. These regions will vary in a predictable way for other tau isoforms (e.g., 2N3R, 1NR4, 1N3R, 0N4R, and 0N3R). Accordingly, when amino acid positions are identified relative to tau-441, a skilled artisan will be able to determine the corresponding amino acid position for the other isoforms.

The term “N-terminal tau,” as used herein, refers to a tau protein, or a plurality of tau proteins, that comprise(s) two or more amino acids of the N-terminus of tau (e.g., amino acids 1-103 of tau-441, etc.).

The term “mid-domain tau,” as used herein, refers to a tau protein, or a plurality of tau proteins, that comprise(s) two or more amino acids of the mid-domain of tau (e.g., amino acids 104-243 of tau-441, etc.).

The term “MTBR tau,” as used herein, refers to a tau protein, or a plurality of tau proteins, that comprise(s) two or more amino acids of the microtubule binding region (MTBR) of tau (e.g., amino acids 244-368 of tau-441, etc.).

The term “C-terminal tau,” as used herein, refers to a tau protein, or a plurality of tau proteins, that comprise(s) two or more amino acids of the C-terminus of tau (e.g., amino acids 369-441of tau-441, etc.).

A “proteolytic peptide of tau” refers to a peptide fragment of a tau protein produced by in vitro proteolytic cleavage. A “tryptic peptide of tau” refers to a peptide fragment of a tau protein produced by in vitro cleavage with trypsin. Tryptic peptides of tau may be referred to herein by their first four amino acids. For instance, “LQTA” refers to the tryptic peptide LQTAPVPMPDLK (SEQ ID NO: 3). Non-limiting examples of other tryptic peptides identified by their first four amino acids include IGST (SEQ ID NO: 2), VQII (SEQ ID NO: 4), LDLS (SEQ ID NO: 5), HVPG (SEQ ID NO: 6), IGSL (SEQ ID NO: 7), VQIV (SEQ ID NO: 9), and TPPS (SEQ ID NO: 10).

A disease associated with tau deposition in the brain is referred to herein as a “tauopathy”. The term “tau deposition” is inclusive of all forms pathological tau deposits including but not limited to neurofibrillary tangles, neuropil threads, and tau aggregates in dystrophic neurites. Tauopathies known in the art include, but are not limited to, progressive supranuclear palsy (PSP), dementia pugilistica, chronic traumatic encephalopathy, frontotemporal dementia and parkinsonism linked to chromosome 17, Lytico-Bodig disease, Parkinson-dementia complex of Guam, tangle-predominant/tangle-only dementia, ganglioglioma and gangliocytoma, meningioangiomatosis, subacute sclerosing panencephalitis, lead encephalopathy, tuberous sclerosis, Hallervorden-Spatz disease, lipofuscinosis, globular glial tauopathy, Pick's disease, corticobasal degeneration (CBD), argyrophilic grain disease (AGD), Frontotemporal lobar degeneration (FTLD), Alzheimer's disease (AD), and frontotemporal dementia (FTD).

Tauopathies are classified by the predominance of tau isoforms found in the pathological tau deposits. Those tauopathies with tau deposits predominantly composed of tau with three MTBRs are referred to as “3R-tauopathies”. Pick's disease is a non-limiting example of a 3R-tauopathy. For clarification, pathological tau deposits of some 3R-tauopathies may be a mix of 3R and 4R tau isoforms with 3R isoforms predominant. Intracellular neurofibrillary tangles (i.e. tau deposits) in brains of subjects with Alzheimer's disease are generally thought to contain both approximately equal amounts of 3R and 4R isoforms. Those tauopathies with tau deposits predominantly composed of tau with four MTBRs are referred to as “4R-tauopathies”. PSP, CBD, and AGD are non-limiting examples of 4R-tauopathies, as are some forms of FTLD. Notably, pathological tau deposits in brains of some subjects with genetically confirmed FTLD cases, such as some V334M and R406W mutation carriers, show a mix of 3R and 4R isoforms.

A clinical sign of a tauopathy may be aggregates of tau in the brain, including but not limited to neurofibrillary tangles, neuropil threads, neuritic plaques. Methods for detecting and quantifying tau aggregates in the brain are known in the art (e.g., tau PET using tau-specific ligands such as [18F]THK5317, [18F]THK5351, [18F]AV1451, [11C]PBB3, [18F]MK-6240, [18F]R0-948, [18F]PI-2620, [18F]GTP1, [18F]PM-PBB3, and [18F]JNJ64349311, [18F]JNJ-067), etc.).

The terms “treat,” “treating,” or “treatment” as used herein, refers to the provision of medical care by a trained and licensed professional to a subject in need thereof. The medical care may be a diagnostic test, a therapeutic treatment, and/or a prophylactic or preventative measure. The object of therapeutic and prophylactic treatments is to prevent or slow down (lessen) an undesired physiological change or disease/disorder. Beneficial or desired clinical results of therapeutic or prophylactic treatments include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented. Accordingly, a subject in need of treatment may or may not have any symptoms or clinical signs of disease.

The phrase “tau therapy” collectively refers to any imaging agent, therapeutic treatment, and/or a prophylactic or preventative measure contemplated for, or used with, subjects at risk of developing a tauopathy, or subjects clinically diagnosed as having a tauopathy. Non-limiting examples of imaging agents include functional imaging agents (e.g. fluorodeoxyglucose, etc.) and molecular imaging agents (e.g., Pittsburgh compound B, florbetaben, florbetapir, flutemetamol, radiolabeled tau-specific ligands, radionuclide-labeled antibodies, etc.). Non-limiting examples of therapeutic agents include cholinesterase inhibitors, N-methyl D-aspartate (NMDA) antagonists, antidepressants (e.g., selective serotonin reuptake inhibitors, atypical antidepressants, aminoketones, selective serotonin and norepinephrine reuptake inhibitors, tricyclic antidepressants, etc.), gamma-secretase inhibitors, beta-secretase inhibitors, anti-Aβ antibodies (including antigen-binding fragments, variants, or derivatives thereof), anti-tau antibodies (including antigen-binding fragments, variants, or derivatives thereof), stem cells, dietary supplements (e.g. lithium water, omega-3 fatty acids with lipoic acid, long chain triglycerides, genistein, resveratrol, curcumin, and grape seed extract, etc.), antagonists of the serotonin receptor 6, p38alpha MAPK inhibitors, recombinant granulocyte macrophage colony-stimulating factor, passive immunotherapies, active vaccines (e.g. CAD106, AF20513, etc.), tau protein aggregation inhibitors (e.g. TRx0237, methylthionimium chloride, etc.), therapies to improve blood sugar control (e.g., insulin, exenatide, liraglutide pioglitazone, etc.), anti-inflammatory agents, phosphodiesterase 9A inhibitors, sigma-1 receptor agonists, kinase inhibitors, phosphatase activators, phosphatase inhibitors, angiotensin receptor blockers, CB1 and/or CB2 endocannabinoid receptor partial agonists, β-2 adrenergic receptor agonists, nicotinic acetylcholine receptor agonists, 5-HT2A inverse agonists, alpha-2c adrenergic receptor antagonists, 5-HT 1A and 1D receptor agonists, Glutaminyl-peptide cyclotransferase inhibitors, selective inhibitors of APP production, monoamine oxidase B inhibitors, glutamate receptor antagonists, AMPA receptor agonists, nerve growth factor stimulants, HMG-CoA reductase inhibitors, neurotrophic agents, muscarinic M1 receptor agonists, GABA receptor modulators, PPAR-gamma agonists, microtubule protein modulators, calcium channel blockers, antihypertensive agents, statins, and any combination thereof.

The term “antibody,” as used herein, is used in the broadest sense and encompasses various antibody and antibody-like structures, including but not limited to full-length monoclonal, polyclonal, and multispecific (e.g., bispecific, trispecific, etc.) antibodies, as well as heavy chain antibodies and antibody fragments provided they exhibit the desired antigen-binding activity. The domain(s) of an antibody that is involved in binding an antigen is referred to as a “variable region” or “variable domain,” and is described in further detail below. A single variable domain may be sufficient to confer antigen-binding specificity. Preferably, but not necessarily, antibodies useful in the discovery are produced recombinantly. Antibodies may or may not be glycosylated, though glycosylated antibodies may be preferred. An “isolated” antibody is one which has been separated from a component of its natural environment. In some embodiments, an antibody is purified to greater than 95% or 99% purity as determined by methods known in the art.

The terms “full length antibody” and “intact antibody” may be used interchangeably, and refer to an antibody having a structure substantially similar to a native antibody structure or having heavy chains that contain an Fc region as defined herein. The basic structural unit of a native antibody comprises a tetramer. Each tetramer is composed of two identical pairs of polypeptide chains, each pair having one “light” chain (about 25 kDa) and one “heavy” chain (about 50-70 kDa). Light chains are classified as gamma, mu, alpha, and lambda. Heavy chains are classified as gamma, mu, alpha, delta, or epsilon, and define the antibody's isotype as IgG, IgM, IgA, IgD and IgE, respectively. The amino-terminal portion of each light and heavy chain includes a variable region of about 100 to 110 or more amino acid sequences primarily responsible for antigen recognition (VL and VH, respectively). The carboxy-terminal portion of each chain defines a constant region primarily responsible for effector function. Within light and heavy chains, the variable and constant regions are joined by a “J” region of about 12 or more amino acid sequences, with the heavy chain also including a “D” region of about 10 more amino acid sequences. Intact antibodies are properly cross-linked via disulfide bonds, as is known in the art.

The variable domains of the heavy chain and light chain of an antibody generally have similar structures, with each domain comprising four conserved framework regions (FRs) and three hypervariable regions (HVRs). (See, e.g., Kindt et al. Kuby Immunology, 6^{th} ed., W. H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

“Framework region” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence: FR1-HVR1-FR2-HVR2-FR3-HVR3-FR4. The FR domains of a heavy chain and a light chain may differ, as is known in the art.

The term “hypervariable region” or “HVR” as used herein refers to each of the regions of a variable domain which are hypervariable in sequence (also commonly referred to as “complementarity determining regions” or “CDR”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). As used herein, “an HVR derived from a variable region” refers to an HVR that has no more than two amino acid substitutions, as compared to the corresponding HVR from the original variable region. Exemplary HVRs herein include: (a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987)); (b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); (c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and (d) combinations of (a), (b), and/or (c), as defined below for various antibodies of this disclosure. Unless otherwise indicated, HVR residues and other residues in the variable domain (e.g., FR residues) are numbered herein according to Kabat et al., supra.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

A “variant Fc region” comprises an amino acid sequence that can differ from that of a native Fc region by virtue of one or more amino acid substitution(s) and/or by virtue of a modified glycosylation pattern, as compared to a native Fc region or to the Fc region of a parent polypeptide. In an example, a variant Fc region can have from about one to about ten amino acid substitutions, or from about one to about five amino acid substitutions in a native sequence Fc region or in the Fc region of the parent polypeptide. The variant Fc region herein may possess at least about 80% homology, at least about 90% homology, or at least about 95% homology with a native sequence Fc region and/or with an Fc region of a parent polypeptide.

An “antibody fragment” refers to a molecule other than an intact antibody that comprises a portion of an intact antibody that binds the antigen to which the intact antibody binds. Non-limiting examples of antibody fragments include but are not limited to Fv, Fab, Fab′, Fab′-SH, F(ab′)2; single-chain forms of antibodies and higher order variants thereof; single-domain antibodies, and multispecific antibodies formed from antibody fragments.

Single-chain forms of antibodies, and their higher order forms, may include, but are not limited to, single-domain antibodies, single chain variant fragments (scFvs), divalent scFvs (di-scFvs), trivalent scFvs (tri-scFvs), tetravalent scFvs (tetra-scFvs), diabodies, and triabodies and tetrabodies. ScFv's are comprised of heavy and light chain variable regions connected by a linker. In most instances, but not all, the linker may be a peptide. A linker peptide is preferably from about 5 to 30 amino acids in length, or from about 10 to 25 amino acids in length. Typically, the linker allows for stabilization of the variable domains without interfering with the proper folding and creation of an active binding site. In preferred embodiments, a linker peptide is rich in glycine, as well as serine or threonine. ScFvs can be used to facilitate phage display or can be used for flow cytometry, immunohistochemistry, or as targeting domains. Methods of making and using scFvs are known in the art. ScFvs may also be conjugated to a human constant domain (e.g. a heavy constant domain is derived from an IgG domain, such as IgG1, IgG2, IgG3, or IgG4, or a heavy chain constant domain derived from IgA, IgM, or IgE). Diabodies, triabodies, and tetrabodies and higher order variants are typically created by varying the length of the linker peptide from zero to several amino acids. Alternatively, it is also well known in the art that multivalent binding antibody variants can be generated using self-assembling units linked to the variable domain.

A “single-domain antibody” refers to an antibody fragment consisting of a single, monomeric variable antibody domain.

Multispecific antibodies include bi-specific antibodies, tri-specific, or antibodies of four or more specificities. Multispecific antibodies may be created by combining the heavy and light chains of one antibody with the heavy and light chains of one or more other antibodies. These chains can be covalently linked.

“Monoclonal antibody” refers to an antibody that is derived from a single copy or clone, including e.g., any eukaryotic, prokaryotic, or phage clone. “Monoclonal antibody” is not limited to antibodies produced through hybridoma technology. Monoclonal antibodies can be produced using hybridoma techniques well known in the art, as well as recombinant technologies, phage display technologies, synthetic technologies or combinations of such technologies and other technologies readily known in the art. Furthermore, the monoclonal antibody may be labeled with a detectable label, immobilized on a solid phase and/or conjugated with a heterologous compound (e.g., an enzyme or toxin) according to methods known in the art.

A “heavy chain antibody” refers to an antibody that consists of two heavy chains. A heavy chain antibody may be an IgG-like antibody from camels, llamas, alpacas, sharks, etc., or an IgNAR from a cartiliaginous fish.

A “humanized antibody” refers to a non-human antibody that has been modified to reduce the risk of the non-human antibody eliciting an immune response in humans following administration but retains similar binding specificity and affinity as the starting non-human antibody. A humanized antibody binds to the same or similar epitope as the non-human antibody. The term “humanized antibody” includes an antibody that is composed partially or fully of amino acid sequences derived from a human antibody germline by altering the sequence of an antibody having non-human hypervariable regions (“HVR”). The simplest such alteration may consist simply of substituting the constant region of a human antibody for the murine constant region, thus resulting in a human/murine chimera which may have sufficiently low immunogenicity to be acceptable for pharmaceutical use. Preferably, the variable region of the antibody is also humanized by techniques that are by now well known in the art. For example, the framework regions of a variable region can be substituted by the corresponding human framework regions, while retaining one, several, or all six non-human HVRs. Some framework residues can be substituted with corresponding residues from a non-human VL domain or VH domain (e.g., the non-human antibody from which the HVR residues are derived), e.g., to restore or improve specificity or affinity of the humanized antibody. Substantially human framework regions have at least about 75% homology with a known human framework sequence (i.e. at least about 75%, at least about 80%, at least about 85%, at least about 90%, at least about 95%, or at least about 99% sequence identity). HVRs may also be randomly mutated such that binding activity and affinity for the antigen is maintained or enhanced in the context of fully human germline framework regions or framework regions that are substantially human. As mentioned above, it is sufficient for use in the methods of this discovery to employ an antibody fragment. Further, as used herein, the term “humanized antibody” refers to an antibody comprising a substantially human framework region, at least one HVR from a nonhuman antibody, and in which any constant region present is substantially human. Substantially human constant regions have at least about 90% with a known human constant sequence (i.e. about 90%, about 95%, or about 99% sequence identity). Hence, all parts of a humanized antibody, except possibly the HVRs, are substantially identical to corresponding pairs of one or more germline human immunoglobulin sequences.

If desired, the design of humanized immunoglobulins may be carried out as follows, or using similar methods familiar to those with skill in the art (for example, see Almagro, et al. Front. Biosci. 2008, 13(5):1619-33). A murine antibody variable region is aligned to the most similar human germ line sequences (e.g. by using BLAST or similar algorithm). The CDR residues from the murine antibody sequence are grafted into the similar human “acceptor” germ line. Subsequently, one or more positions near the CDRs or within the framework (e.g., Vernier positions) may be reverted to the original murine amino acid in order to achieve a humanized antibody with similar binding affinity to the original murine antibody. Typically, several versions of humanized antibodies with different reversion mutations are generated and empirically tested for activity. The humanized antibody variant with properties most similar to the parent murine antibody and the fewest murine framework reversions is selected as the final humanized antibody candidate.

### II. Epitope Binding Agents That Specifically Bind to Tau

The present disclosure encompasses epitope binding agents that specifically bind to human tau. An epitope binding agent that specifically binds to human tau refers to an isolated peptide, protein or nucleic acid that binds to tau with an affinity constant or affinity of interaction (KD) between about 0.1 pM to about 10 μM, preferably about 0.1 pM to about 1 μM, more preferably about 0.1 pM to about 100 nM. The term “affinity” refers to a measure of the strength of the binding of an individual epitope with an antibody's antigen binding site. Methods for determining the affinity of an epitope-binding agent for an antigen are known in the art, and further detailed in the Examples.

In one aspect, an epitope binding agent that specifically binds to human tau is an antibody that specifically binds to human tau. The term “anti-tau antibody” and the term “an antibody that specifically binds to human tau” are interchangeable. In some embodiments, an anti-tau antibody is an IgG subtype. In further embodiments, an anti-tau antibody is an IgG1 subtype. In still further embodiments, an anti-tau antibody is an IgG4 subtype. In each of the above embodiments, an anti-tau antibody of this disclosure may or may not have a variant Fc region. For example, an Fc region can be modified to have increased or decreased affinity for an Fc receptor on a microglial cell and/or an altered glycosylation pattern.

In another aspect, an epitope binding agent that specifically binds to human tau is an antibody mimetic that specifically binds to human tau. An “antibody mimetic” refers to a polypeptide or a protein that can specifically bind to an antigen but is not structurally related to an antibody. Antibody mimetics have a mass of about 3 kDa to about 20 kDa. Non-limiting examples of antibody mimetics are affibody molecules, affilins, affimers, alphabodies, anticalins, avimers, DARPins, and monobodies.

In another aspect, an epitope binding agent that specifically binds to human tau is an aptamer that specifically binds to human tau. Aptamers are a class of small nucleic acid ligands that are composed of RNA or single-stranded DNA oligonucleotides and have high specificity and affinity for their targets. Aptamers interact with and bind to their targets through structural recognition, a process similar to that of an antigen-antibody reaction. Aptamers have a lower molecular weight than antibodies, typically about 8-25 kDa.

Methods for generating antibodies, antibody mimetics, and aptamers are well known in the art.

Epitope binding agents that specifically bind to human tau that are useful herein include those which are suitable for administration to a subject in a therapeutic amount, as well as those which are suitable for in vivo and/or in vitro diagnostic applications. Epitope binding agents disclosed herein may be conjugated to therapeutic agents, prodrugs, imaging agents, targeting agents, peptides, proteins, enzymes, viruses, biological response modifiers, pharmaceutical agents, or PEG.

In addition to binding to tau with a specific affinity, useful anti-tau epitope binding agents can be described or specified in terms of the epitope(s) that they recognize or bind. The portion of a target polypeptide that specifically interacts with the antigen binding domain of an epitope binding agent is an “epitope.” Tau can comprise any number of epitopes, depending on the source of the protein (e.g. mouse, rat, cynomolgus monkey, human, etc.), isoform (e.g. 0N3R, 0N4R, 1N3R, 1N4R, 2N3R, 2N4R), conformational state of the isoform (e.g., fibrillar, aggregated, insoluble, soluble, monomeric, oligomeric, oxidized, post-translationally modified, truncated, etc.) and location of the isoform (e.g., intracellular, extracellular, CNS, periphery, etc.).

In some embodiments, epitope binding agents of the present disclosure specifically bind human tau and recognize an epitope within an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK). To clarify, “an epitope within SEQ ID NO: 3” includes epitopes 12, 11, 10, 9, 8, 7, 6, 5, 4, etc. amino acids in length.

In some embodiments, an anti-tau epitope binding agent of the present disclosure specifically binds human tau and recognizes an epitope comprising four or more continuous amino acids of an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK).

In some embodiments, an anti-tau epitope binding agent of the present disclosure specifically binds human tau and recognizes an epitope comprising at least four continuous amino acid residues of an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK). In some embodiments, an anti-tau epitope binding agent of the present disclosure specifically binds human tau and recognizes an epitope comprising at least five continuous amino acid residues of an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK). In some embodiments, an anti-tau epitope binding agent of the present disclosure specifically binds human tau and recognizes an epitope comprising at least six continuous amino acid residues of an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK). In some embodiments, an anti-tau epitope binding agent of the present disclosure specifically binds human tau and recognizes an epitope comprising at least seven continuous amino acid residues of an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK). In some embodiments, an anti-tau epitope binding agent of the present disclosure specifically binds human tau and recognizes an epitope comprising at least eight continuous amino acid residues of an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK). In some embodiments, an anti-tau epitope binding agent of the present disclosure specifically binds human tau and recognizes an epitope comprising at least nine continuous amino acid residues of an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK). In some embodiments, an anti-tau epitope binding agent of the present disclosure specifically binds human tau and recognizes an epitope comprising at least ten continuous amino acid residues of an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK). In some embodiments, an anti-tau epitope binding agent of the present disclosure specifically binds human tau and recognizes an epitope comprising at least eleven continuous amino acid residues of an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK). In some embodiments, an anti-tau epitope binding agent of the present disclosure specifically binds human tau and recognizes an epitope comprising at least twelve continuous amino acid residues of an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK). In some embodiments, an anti-tau epitope binding agent of the present disclosure specifically binds human tau and recognizes an epitope comprising at least thirteen continuous amino acid residues of an amino acid sequence selected from the group consisting of SEQ ID NO: 6 (HVPGGGSVQIVYKPVDLSK) and SEQ ID NO: 7 (IGSLDNITHVPGGGNK). In some embodiments, an anti-tau epitope binding agent of the present disclosure specifically binds human tau and recognizes an epitope comprising at least fourteen continuous amino acid residues of an amino acid sequence selected from the group consisting of SEQ ID NO: 6 (HVPGGGSVQIVYKPVDLSK) and SEQ ID NO: 7 (IGSLDNITHVPGGGNK). In some embodiments, an anti-tau epitope binding agent of the present disclosure specifically binds human tau and recognizes an epitope comprising at least fifteen continuous amino acid residues of an amino acid sequence selected from the group consisting of SEQ ID NO: 6 (HVPGGGSVQIVYKPVDLSK) and SEQ ID NO: 7 (IGSLDNITHVPGGGNK). In some embodiments, an anti-tau epitope binding agent of the present disclosure specifically binds human tau and recognizes an epitope comprising at least sixteen continuous amino acid residues of an amino acid sequence selected from the group consisting of SEQ ID NO: 6 (HVPGGGSVQIVYKPVDLSK) and SEQ ID NO: 7 (IGSLDNITHVPGGGNK). In some embodiments, an anti-tau epitope binding agent of the present disclosure specifically binds human tau and recognizes an epitope comprising at least seventeen continuous amino acid residues of SEQ ID NO: 6 (HVPGGGSVQIVYKPVDLSK). In some embodiments, an anti-tau epitope binding agent of the present disclosure specifically binds human tau and recognizes an epitope comprising at least eighteen continuous amino acid residues of SEQ ID NO: 6 (HVPGGGSVQIVYKPVDLSK). In some embodiments, an anti-tau epitope binding agent of the present disclosure specifically binds human tau and recognizes an epitope comprising at least nineteen continuous amino acid residues of SEQ ID NO: 6 (HVPGGGSVQIVYKPVDLSK).

In each of the above embodiments, the epitope can be a linear epitope or a conformational epitope, and in both instances can include non-polypeptide elements, e.g., an epitope can include a carbohydrate, lipid side chain, phosphate, etc.

In certain embodiments the epitope(s) to which anti-tau epitope binding agents of this disclosure bind may be enriched in, or unique to, a subset of tau isoforms, preferentially pathological tau isoforms. Consequently, anti-tau epitope binding agents of this disclosure may preferentially bind one or more tau isoform (“a subset of tau isoforms”). An epitope binding agent that preferentially binds to a subset of tau isoforms binds to that isoform/those isoforms more readily than it would a different tau isoform not in the subset, as assessed in an in vitro binding assay. As a non-limiting example, an epitope binding agent can be considered to bind a tau isoform preferentially if the binding half maximal concentration (EC₅₀) of the epitope binding agent for that isoform is at least about 5-fold, 10-fold, 50-fold, or 100-fold less than EC5o for the other isoforms as measured in an ELISA or similar assay. Alternatively, an epitope binding agent can be described as not preferentially binding a given tau isoform if the EC₅₀ for the epitope binding agent for that tau isoform and the other tau isoforms vary by less 5-fold, or less than 10-fold. For instance, in some embodiments, anti-tau epitope binding agents of the present disclosure may preferentially bind truncated tau isoforms over full-length isoforms (i.e., 0N3R, 0N4R, 1N3R, 1N4R, 2N3R, 2N4R). Alternatively, or in addition, in some embodiments, anti-tau epitope binding agents of the present disclosure may not specifically bind full-length tau isoforms. Alternatively, or in addition, in some embodiments, anti-tau epitope binding agents of the present disclosure may preferentially bind mid-domain independent MTBR tau species, for example, mid-domain independent MTBR tau comprising SEQ ID NO: 3 (LQTAPVPMPDLK), SEQ ID NO: 6 (HVPGGGSVQIVYKPVDLSK), SEQ ID NO: 7 (IGSLDNITHVPGGGNK), or any combination thereof.

In a specific example, an anti-tau epitope binding agent of the present disclosure specifically binds human tau and recognizes a neo-epitope of tau produced by a pathological process. A “neo-epitope of tau” refers to an epitope that is generated by modification of the original epitope. As noted above, the term “tau,” as used herein, refers to a plurality of isoforms encoded by the gene MAPT, as well as species thereof that are C-terminally truncated in vivo, N-terminally truncated in vivo, post-translationally modified in vivo, or any combination thereof. N-terminally truncated tau isoforms and C-terminally truncated isoforms may therefore contain neo-epitopes that do not exist in a full-length isoform (i.e., 0N3R, 0N4R, 1N3R, 1N4R, 2N3R, 2N4R).

Non-limiting examples of subsets of N-terminally truncated tau isoforms (referred to as mid-domain independent MTBR tau species) produced by pathological processes, as evidenced by their unique correlation with Alzheimer's disease pathology, are detailed in Example 1. Without wishing to be bound by theory, one example of a neo-epitope may be an N-terminal free end of a mid-domain independent MTBR tau species that is generated in vivo by cleavage of the peptide bond between amino acids 242 and 243 of tau-441 (or the same residues for other full-length isoforms), or cleavage of a peptide bond proximal to amino acid 243 of tau-441 (or the same residues for other full-length isoforms). Another example of a neo-epitope may be a C-terminal free end of tau species that is generated in vivo by cleavage of the peptide bond between amino acids 254 and 255 of tau-441 (or the same residues for other full-length isoforms) or cleavage of a peptide bond proximal to amino acid 254 of tau-441 (or the same residues for other full-length isoforms). A skilled artisan will appreciate a number of conformational or linear neo-epitopes comprising at least four continuous amino acid residues of an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK), may be therefore be produced by pathological processing of tau.

An anti-tau epitope binding agent of the present disclosure is useful in an immunoassay. Non-limiting examples of an immunoassay comprising an anti-tau epitope binding agent of the present disclosure include an ELISA, a lateral flow assay, a sandwich immunoassay, a radioimmunoassay, an immunoblot or Western blot, flow cytometry, immunohistochemistry, and an array. A lateral flow assay may be a device intended to detect the presence (or absence) of a target analyte in sample. An anti-tau epitope binding agent of the present disclosure for may be conjugated to a label. The term “label”, as used herein, refers to any substance attached to an antibody, or other substrate material, in which the substance is detectable by a detection method. Non-limiting examples of suitable labels include luminescent molecules, chemiluminescent molecules, fluorochromes, fluorescent quenching agents, colored molecules, radioisotopes, scintillants, biotin, avidin, stretpavidin, protein A, protein G, antibodies or fragments thereof, polyhistidine, Ni2+, Flag tags, myc tags, heavy metals, and enzymes (including alkaline phosphatase, peroxidase, glucose oxidase and luciferase). Methods of detecting and measuring an amount of an epitope binding agent-polypeptide complex based on the detection of a label or marker are well known in the art.

Immunoassays can be run in a number of different formats. Generally speaking, immunoassays can be divided into two categories: competitive immmunoassays and non-competitive immunoassays. In a competitive immunoassay, an unlabeled analyte in a sample competes with labeled analyte to bind an epitope binding agent. Unbound analyte is washed away and the bound analyte is measured. In a non-competitive immunoassay, the epitope binding agent is labeled, not the analyte. Non-competitive immunoassays may use one epitope binding agent (e.g. the capture antibody is labeled) or more than one epitope binding agent (e.g. at least one capture antibody which is unlabeled and at least one “capping” or detection antibody which is labeled). Suitable labels are described above.

In alternative embodiments, an epitope binding agent of the disclosure can be used in an array. An array comprises at least one address, wherein at least one address of the array has disposed thereon an anti-tau epitope binding agent of the present disclosure. Arrays may comprise from about 1 to about several hundred thousand addresses. Several substrates suitable for the construction of arrays are known in the art, and one skilled in the art will appreciate that other substrates may become available as the art progresses. Non-limiting examples of suitable surfaces include microtitre plates, test tubes, beads, resins, and other polymers. In some embodiments, the array comprises at least one anti-tau epitope binding agent of the present disclosure attached to the substrate is located at one or more spatially defined addresses of the array. For example, an array may comprise at least one, at least two, at least three, at least four, or at least five an anti-tau epitope binding agents of the present disclosure, each binding agent recognizing the same or different tau epitopes, and each eptitope binding agent may be may be at one, two, three, four, five, six, seven, eight, nine, ten or more spatially defined addresses.

### III. Compositions

Still another aspect of the present disclosures provides compositions comprising an epitope binding agent disclosed in Section II. Compositions of the present disclosure may further comprise compatible carriers, buffers, pH adjusting agents, stabilizing agents, preservatives, and the like. The choice of these additional components can and will vary depending upon in the intended use of the compositions, e.g., in vitro diagnostic, in vivo diagnostic, therapeutic, etc.

In some embodiments, an epitope binding agent of Section II may be admixed with at least one pharmaceutically acceptable carrier or excipient resulting in a pharmaceutical composition, which is capably and effectively administered (given) to a living subject, such as to a suitable subject (e.g., a subject in need of treatment, a subject in need of a diagnostic test for tau, etc.). Methods of preparing pharmaceutical formulations comprising anti-tau antibodies and other epitope binding agents of Section II to a subject in need thereof are well known to or are readily determined by those skilled in the art.

Pharmaceutical compositions for effective administration are deliberately designed to be appropriate for the selected mode of administration, and pharmaceutically acceptable excipients such as compatible carriers, dispersing agents, buffers, surfactants, preservatives, solubilizing agents, isotonicity agents, stabilizing agents and the like are used as appropriate.

Non-limiting examples of pharmaceutically acceptable carriers, include physiological saline, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, wool fat or oa combination thereof.

Prevention of the action of microorganisms can be achieved by various antibacterial and antifungal agents, for example, parabens, chlorobutanol, phenol, ascorbic acid, thimerosal and the like. In many cases, isotonic agents can be included, for example, sugars, polyalcohols, such as mannitol, sorbitol, or sodium chloride in the composition.

Prolonged absorption of the injectable compositions can be brought about by including in the composition an agent which delays absorption, for example, aluminum monostearate and gelatin.

Compositions disclosed herein can be frozen or lyophilized for storage and reconstituted in a suitable sterile carrier prior to use.

In some embodiments, anti-tau antibodies or other epitope binding agents of Section II may be formulated for parenteral administration. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like. Parenteral formulations can be a single bolus dose, an infusion or a loading bolus dose followed with a maintenance dose. These compositions can be administered at specific fixed or variable intervals, e.g., once a day, or on an “as needed” basis.

Certain pharmaceutical compositions, as disclosed herein, can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of an epitope binding agent of Section II to be combined with the carrier materials to produce a single dosage form will vary depending upon the host treated and the particular mode of administration. The composition can be administered as a single dose, multiple doses or over an established period of time in an infusion. Dosage regimens also can be adjusted to provide the optimum desired response (e.g., a therapeutic or prophylactic response).

Also provided are kits. Such kits can include an epitope or epitope binding agent described herein and, in certain embodiments, instructions for administration or use. Such kits can facilitate performance of the methods described herein. When supplied as a kit, the different components of the composition can be packaged in separate containers and admixed immediately before use. Components include, but are not limited to compositions and pharmaceutical formulations comprising an epitope binding agent composition, as described herein, an isolated epitope binding agent as described herein, or an immunoassay comprising an epitope binding agent disclosed herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

### IV. Treatment Methods

Still another aspect of the present disclosures provides a method of treating a tauopathy in a subject in need thereof. In general, the method comprises administering to the subject a therapeutically effective amount of an epitope binding agent of Section II either alone or in combination with at least on additional therapeutic agent. In some embodiments, the tauopathy is a 3R tauopathy. In other embodiments, the tauopathy is a 4R tauopathy. In still other embodiments, the tauopathy is a 3R/4R tauopathy. In a specific embodiment, the tauopathy is Alzheimer's disease.

Treating a tauopathy may stabilize one or more aspect of a subject's disease, may improve one or more aspect of a subject's disease, may prevent a subject from developing a clinical sign or symptom of a tauopathy, or any combination thereof. Accordingly, those in need of treatment include subjects already with the disease, as well as those prone to have the disease or those in which the disease is to be prevented. Accordingly, a subject in need of treatment may or may not have any symptoms or clinical signs of disease.

In general, the subject will be a human. Without departing from the scope of the invention, however, other mammalian subjects may be used. Suitable mammalian subjects include; companion animals, such as cats and dogs; livestock animals, such as cows, pigs, horses, sheep, and goats; zoo animals; and research animals, such as non-human primates and rodents. In embodiments where the subject is a human, and the epitope binding agent is an antibody, the antibody is adapted for administration to a living human subject (e.g. humanized).

In one embodiment, the disclosure provides a method of preventing the progression, or slowing the rate of progression, of a tauopathy in a subject in need thereof, the method comprising administering to the subject a therapeutically effective amount of an epitope binding agent of Section II. Progression of a tauopathy can be evaluated is typically indicated by a worsening of a clinical symptom and/or a clinical sign associated with the tauopathy, and can be assessed by performing repeated measurements of the clinical symptom and/or clinical sign over time (e.g., weeks, months, years). In a specific embodiment, disease progression is assessed by quantitative measurements of tau aggregates and/or disease specific tau isoforms in the brain using a tau-specific PET tracer (e.g., tau PET AV-1451, a radiolabeled antibody, etc.). In another specific embodiment, disease progression is assessed by quantitative measurement, in blood or CSF, of biomarkers of tau aggregates in the brain. Non-limiting examples of biomarkers of tau aggregates in the brain that can be measured in the periphery (e.g., CSF, blood) include phosphorylated tau species including but not limited to tau phosphorylated at threonine 217 (P-tau217), serine 205 (P-tau205), and at threonine 181 (P-tau181), wherein all numbering is based on tau-441, as well as mid-domain-independent MTBR tau species detailed in Example 1.

In another embodiment, the disclosure provides a method of reducing a tauopathy-related pathology in a subject in need thereof, the method comprising administering to the subject a therapeutically effective amount of an epitope binding agent of Section II. The tauopathy-related pathology may be plasma, CSF, or PET biomarkers of tau aggregates in the brain. Alternatively, or in addition, the tauopathy-related pathology may be tau-independent, for example, other plasma, CSF, PET or MRI biomarkers of neuronal cell death or dysfunction, amyloid deposition, other pathological protein aggregates in the brain (e.g., TDP-43, alpha-synuclein), etc.

In another embodiment, the disclosure provides a method of reducing pathological tau deposits in a brain of a subject in need thereof, the method comprising administering to the subject a therapeutically effective amount of an epitope binding agent of Section II. The pathological tau deposits may be neufibrillary tangles, neutropil threads, neuritic plaques, or any combination thereof. In a specific example, pathological tau deposits in a brain of subject may be quantified by tau PET AV-1451 SUVR, as described in Example 1.

The therapeutically effective amount of the epitope binding agent is typically formulated in a pharmaceutical composition further comprising a pharmaceutically acceptable carrier and/or excipient, as detailed in Section III. Administration of an epitope binding agent of Section II, or a pharmaceutical composition of Section III, is performed using standard effective techniques, include peripherally (i.e., not by administration into the central nervous system) or locally to the central nervous system. Peripheral administration includes but is not limited to intravenous, intraperitoneal, subcutaneous, pulmonary, transdermal, intramuscular, intranasal, buccal, sublingual, or suppository administration. Local administration, including directly into the central nervous system (CNS) includes but is not limited to via a lumbar, intraventricular or intraparenchymal catheter or using a surgically implanted controlled release formulation.

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners.

The concentration of epitope binding agent in formulations to be administered is an effective amount and ranges from as low as about 0.1% by weight to as much as about 15 or about 20% by weight and will be selected primarily based on fluid volumes, viscosities, and so forth, in accordance with the particular mode of administration selected if desired. A typical composition for injection to a living subject may contain about 1 mL sterile buffered water of phosphate buffered saline and about 1-1000 mg of any one of or a combination of the epitope binding agents disclosed herein. The formulation may be sterile filtered after making the formulation, or otherwise made microbiologically acceptable. A typical composition for intravenous infusion may have volumes between 1-250 mL of fluid, such as sterile Ringer's solution, and 1-100 mg per ml, or more of an epitope binding agent of Section II. As noted in Section II, epitope binding agents disclosed herein can be frozen or lyophilized for storage and reconstituted in a suitable sterile carrier prior to use. Lyophilization and reconstitution may lead to varying degrees of activity loss (e.g. with conventional immune globulins, IgM antibodies tend to have greater activity loss than IgG antibodies). Dosages administered are effective dosages and may have to be adjusted to compensate. The pH of the formulations generally pharmaceutical grade quality, will be selected to balance stability of the epitope binding agent (chemical and physical) and comfort to the subject when administered. Generally, a pH between 4 and 8 is tolerated. Doses may vary from individual to individual based on size, weight, and other physiobiological characteristics of the individual receiving the successful administration.

As used herein, the term “therapeutically effective amount” means an amount of a substance (e.g. an epitope binding agent of Section II) that leads to measurable and beneficial effects for the subject administered the substance, i.e., significant efficacy. The therapeutically effective amount or dose of compound administered according to this discovery will be determined using standard clinical techniques and may be by influenced by the circumstances surrounding the case, including the epitope binding agent administered, the route of administration, and the status of the symptoms being treated, among other considerations. A typical dose may contain from about 0.01 mg/kg to about 100 mg/kg of an anti-tau antibody described herein. Doses can range from about 0.05 mg/kg to about 50 mg/kg, more preferably from about 0.1 mg/kg to about 25 mg/kg. The frequency of dosing may be daily or once, twice, three times or more per week or per month, as needed as to effectively treat the symptoms.

The epitope binding agent may be co-administered with at least one additional therapeutic agent. Exemplary additional therapeutic agents include, but are not limited , cholinesterase inhibitors (such as donepezil, galantamine, rovastigmine, and tacrine), NMDA receptor antagonists (such as memantine), amyloid beta peptide aggregation inhibitors, antioxidants, gamma-secretase modulators, nerve growth factor (NGF) mimics or NGF gene therapy, PPARy agonists, HMS-CoA reductase inhibitors (statins), ampakines, calcium channel blockers, GABA receptor antagonists, glycogen synthase kinase inhibitors, intravenous immunoglobulin, muscarinic receptor agonists, nicotinic receptor modulators, active or passive amyloid beta peptide immunization, phosphodiesterase inhibitors, serotonin receptor antagonists and anti-amyloid beta peptide antibodies or further anti-tau antibodies. Additional exemplary neurological drugs may be selected from a growth hormone or neurotrophic factor; examples include but are not limited to brain-derived neurotrophic factor (BDNF), nerve growth factor (NGF), neurotrophin-4/5, fibroblast growth factor (FGF)-2 and other FGFs, neurotrophin (NT)-3, erythropoietin (EPO), hepatocyte growth factor (HGF), epidermal growth factor (EGF), transforming growth factor (TGF)-al ha, TGF-beta, vascular endothelial growth factor (VEGF), interleukin-1 receptor antagonist (IL-Ira), ciliary neurotrophic factor (CNTF), glial-derived neurotrophic factor (GDNF), neurturin, platelet-derived growth factor (PDGF), heregulin, neuregulin, artemin, persephin, interleukins, glial cell line derived neurotrophic factor (GFR), granulocyte-colony stimulating factor (CSF), granulocyte-macrophage-CSF, netrins, cardiotrophin-1, hedgehogs, leukemia inhibitory factor (LIF), midkine, pleiotrophin, bone morphogenetic proteins (BMPs), netrins, saposins, semaphorins, and stem cell factor (SCF). In certain embodiments, the at least one additional therapeutic agent is selected for its ability to mitigate one or more side effects of the neurological drug. Such combination therapies noted above encompass combined administration (where two or more therapeutic agents are included in the same or separate formulations), and separate administration, in which case, administration of the epitope binding agent can occur prior to, simultaneously, and/or following, administration of the additional therapeutic agent and/or adjuvant. Epitope binding agents of Section II can also be used in combination with other interventional therapies such as, but not limited to, radiation therapy, behavioral therapy, or other therapies known in the art and appropriate for the tauopathy to be treated or prevented.

The timing of administration of the treatment relative to the disease itself and duration of treatment will be determined by the circumstances surrounding the case. Duration of treatment could range from a single dose administered on a one-time basis to a life-long course of therapeutic treatments.

Although the foregoing methods appear the most convenient and most appropriate and effective for administration of proteins such as humanized antibodies, by suitable adaptation, other effective techniques for administration, such as intraventricular administration, transdermal administration and oral administration may be employed provided proper formulation is utilized herein. In addition, it may be desirable to employ controlled release formulations using biodegradable films and matrices, or osmotic mini-pumps, or delivery systems based on dextran beads, alginate, or collagen.

### V. Diagnostic Methods

Still another aspect of the present disclosures provides epitope binding agents of Section II conjugated to a detectable signal (i.e., a measurable substance, or a substance that generates a measurable signal). Non-limiting examples include various enzymes, prosthetic groups, fluorescent materials, luminescent materials, bioluminescent materials, radioactive materials, positron emitting metals using various positron emission tomographies, and nonradioactive paramagnetic metal ions. See, e.g., U.S. Pat. No. 4,741,900 for metal ions which can be conjugated to antibodies for use as diagnostics according to the disclosure. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc. The signal generated by the agent can be measured, for example, by single-photon emission computed tomography (SPECT) or positron emission tomography (PET).

Epitope binding agents of Section II conjugated to a detectable signal may be used diagnostically. For example, the present disclosure also provides the use of the epitope binding agents disclosed herein conjugated to a detectable signal for measuring the amount of tau deposition in a brain of a subject, assessing disease progression in a subject who may or may not be receiving treatment for a tauopathy, determining the efficacy of a given treatment and/or prevention regimen. When a subject is receiving treatment, the treatment can be adjusted based on the measurement of disease progression (e.g., change to a new treatment appropriate for the disease state, adjust the dosage of the current treatment, etc.).

In some embodiments, the present disclosure encompasses a method of assessing disease progression in a subject being treated for a tauopathy, the method comprising: (a) administering at a first time to the subject an epitope binding agent disclosed herein (“anti-tau agent”) that is labeled with an agent that generates a measurable signal as described herein (i.e. “labeled anti-tau agent”), wherein the signal is measured in the subject following the first administration; (b) administering at a second time the labeled anti-tau agent, wherein the second administration occurs after the first administration (e.g., days, week, months), wherein the signal is measured in the patient following the second administration; and (c) assessing disease progression in the patient based on a change in the signal measured between the first and second administration; wherein an increase in the signal indicates progression of the tauopathy in the patient. In certain embodiments, the subject is being treated with the same anti-tau agent, but in an unlabeled form. In certain embodiments, the subject is being treated with an anti-tau antibody that competitively inhibits the labeled anti-tau agent binding to human tau. In certain embodiments, the subject is being treated with an anti-tau antibody that does not competitively inhibit the labeled anti-tau agent binding to human tau. In certain embodiments, the therapy is with an anti-Aβ antibody, an anti-tau antibody, a gamma-secretase inhibitor, a beta-secretase inhibitor, a cholinesterase inhibitor, an NMDA receptor antagonist, or other drugs known in the art.

In some embodiments, the present disclosure encompasses a method of assessing disease progression in a subject being treated for a tauopathy, the method comprising: (a) administering at a first time to the subject an epitope binding agent disclosed herein (“anti-tau agent”) that is labeled with an agent that generates a measurable signal as described herein (i.e. “labeled anti-tau agent”), wherein the signal is measured in the subject following the first administration; (b) assessing the disease state in the subject upon review of a comparison of the signal measured in the subject to the signal measured following administration of the labeled anti-tau agent to one or more control subjects; wherein an increase in the signal generated in the patient relative to the control subject correlates with an increase in tauopathy-related pathology; and (c) treating the patient with a therapy appropriate for the patient's disease state. A “control subject” refers to any normal healthy subject, a subject with different degrees of disease, or even the actual test subject at an earlier stage of disease. In certain embodiments, the therapy is the same anti-tau agent, but in an unlabeled form. In certain embodiments, the therapy is an anti-tau antibody that competitively inhibits the labeled anti-tau agent binding to human tau. In certain embodiments, the therapy is an anti-tau antibody that does not competitively inhibit the labeled anti-tau agent binding to human tau. In certain embodiments, the therapy is with an anti-Aβ antibody, an anti-tau antibody, a gamma-secretase inhibitor, a beta-secretase inhibitor, a cholinesterase inhibitor, an NMDA receptor antagonist, or other drugs known in the art.

### VI. Methods for Inhibiting a Pathological Tau Species

A further aspect of the present disclosure provides methods for inhibiting pathological tau species. Extracellular pathological tau species transmit pathology from cell to cell. Accordingly, by targeting these spreading species with epitope binding agents disclosed herein, it is possible to slow or halt the progression of tau pathology. Effective epitope bindings agents should neutralize the pathological species present in Alzheimer's disease brains and block their cell-to-cell spread. In general, the method comprises contacting pathological tau species with an epitope binding agent of Section II. The pathological tau species may be tau monomers or tau fibrils. In preferred embodiments, the pathological tau species are derived from human brain tauopathy-related pathology. In an exemplary embodiment, the pathological tau species are derived from Alzheimer's disease human brain pathology.

The method of inhibiting pathological tau species may be conducted in vivo or it may be conducted in vitro. Accordingly, the pathological tau species may be disposed in a subject as detailed above. Inhibition of pathological tau species in an animal model may be measured by assaying inhibition of tau seeding and/or tau spreading after passive immunization with the epitope binding agent. See, for example, Albert et al., Brain, 2019, 142(6): 1736-1750. Alternatively, or in addition, inhibition of pathological tau species may be measured by assaying inhibition of tau seeding and/or tau spreading in cell culture. See, for example, Guo et al., J Biol Chem, 2011, 286(17): 15317-15331; or Kfoury et al., J Biol Chem, 2012, 287(23): 19440-19451.

In general, seeding and/or spreading of pathological tau species in vitro or in vivo may be decreased by at least about 10%. In various embodiments, the seeding and/or spreading may be decreased by about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 70%, about 70% to about 80%, about 80% to about 90%, or about 90% to about 99%. In some embodiments, the seeding and/or spreading may be decreased by at least about 10%, at least about 20%, at least about 30%, at least about 40%, at least about 50%, at least about 60%, at least about 70%, at least about 80%, at least about 90%, or at least about 95%.

In vitro assays may also be used to determine the optimal inhibitory concentration (or IC₅₀) of an epitope binding agent of Section II. That is, a dose-response curve may be generated in which the concentration of the epitope binding agent comprising inhibitory activity for pathological tau is varied such that the optimal inhibitory concentration may be determined.

### EXAMPLES

The following examples illustrate various iterations of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

### Example 1

In this example, the presence of disease-specific MTBR tau species in the extracellular space of the CNS is detailed. The results show that a significant amount of “mid-domain-independent MTBR tau” exists in CSF resulting in a C-terminal fragment (a “C-terminal stub”) that lacks the N-terminus region and at least a substantial portion of the mid-domain region. Moreover, the results show that different regions of CSF MTBR tau stage disease progression and correlate with tau aggregation within the Alzheimer's disease brain. These findings provide new insights into the relationship between MTBR tau in the brain and CSF and support the use disease specific MTRB tau species as therapeutic targets.

Materials: Two different cohorts of human brain samples were used in the experiments of this example—a discovery cohort and a validation cohort. The discovery cohort contained postmortem frozen brain tissue samples from two participants with Alzheimer's disease pathology and two control participants without pathology, which were provided by the Knight ADRC Pathology Core at Washington University School of Medicine. Each sample was classified according to the National Institute on Aging and Alzheimer's Association amyloid stage A3 (ThaI phase) for amyloid deposition and Tau Braak stage VI, B3 for tau aggregation. Samples from each participant were collected from six to ten brain regions including the cerebellum, superior frontal gyrus, frontal pole, temporal, occipital, thalamus, amygdala, pons, parietal and striatum. Additional postmortem frozen brain tissue samples from the parietal lobe were analyzed from 20 participants (eight amyloid-negative and 12 amyloid-positive by CSF Aβ 42/40 ratios) as a validation cohort. The 12 amyloid-positive samples were further divided into clinical groups according to their Clinical Dementia Rating (CDR) scores, and classified as very mild to moderate Alzheimer's disease (amyloid-positive, CDR=0.5-2, n=5) or severe Alzheimer's disease (amyloid-positive, CDR=3, n=7). These human studies were approved by the Washington University Institutional Review Board.

Three different cohorts of human CSF samples were also used in the experiments of this example—a cross-sectional cohort, a longitudinal cohort, and a Tau PET cohort (Table 1A and Table 1B). CSF samples from 100 participants were collected from the amyloid beta (Aβ) stable isotope labeling kinetics (SILK) study (Patterson et al., 2015) for analysis as a cross-sectional cohort. CSF collection was performed as previously described (Patterson et al., 2015). Briefly, CSF was collected at baseline. Next, participants received a leucine bolus infusion over 10 minutes. Six mL of CSF was obtained hourly for 36 hours. CSF aliquots collected at hour 30 were used for MS measurement of tau species in this study. Amyloid status was defined using CSF Aβ 42/40 ratio as previously reported (Patterson et al., 2015). The corresponding cutoff ratio (0.1389) maximized the accuracy in predicting amyloid-positivity as determined by Pittsburgh compound B (PiB) PET. Amyloid groups were further divided into clinical groups according to their CDR scores as shown in Table 1A. From the cross-sectional cohort, 28 participants (14 amyloid-positive and 14 amyloid-negative) were followed for two to nine years to assess the longitudinal trajectory of tau species in CSF. CSF samples were collected and analyzed in the same manner as the cross-sectional cohort. The Tau PET cohort contained thirty-five participants (20 amyloid-positive and 15 amyloid-negative, including 16 participants from longitudinal cohort) who had tau PET AV-1451 standardized uptake value ratio (SUVR) measures within three years from the time of CSF collection. PET scans were performed as previously described (Sato et al., 2018) and the partial-volume correction was performed for SUVR using a regional spread function technique (Su et al., 2015). CSF samples were collected and analyzed in the same manner as the other cohorts.

TABLE 1A
Cross-sectional cohort

| | Cross-sectional cohort (n = 100) | | | | |
|---|---|---|---|---|---|
| Variable | Control | Preclinical AD | Very Mild AD | Mild-Moderate AD | Non-AD CI |
| n | 30 | 18 | 28 | 12 | 12 |
| Age | 71 (5) | 73 (7) | 75 (7) | 72 (8) | 75 (8) |
| Gender (F/M) | 18/12 | 11/7 | 11/17 | 2/10 | 2/10 |
| CDR | 0 | 0 | 0.5 | 1-2 (>1) | 0.5-1 (>0.5) |
| CSF Aβ 42/40 | 0.18 (0.02) | 0.10 (0.02) | 0.09 (0.02) | 0.10 (0.02) | 0.17 (0.02) |
| PiB SUVR | 1.04 (0.11) 27 | 1.99 (0.87) 16 | 3.14 (0.92) 13 | 2.73 (2.10) 2 | 0.98 (0.05) 5 |
| AV45 SUVR | 1.12 (0.42) 16 | 1.85 (0.52) 8 | 2.18 (0.53) 6 | 2.41 (na) 1 | 0.96 (0.15) 2 |
| Amyloid status | negative | positive | positive | positive | negative |
| AV-1451 SUVR | na | na | na | na | na |
| CSF tau level (ng/mL) | | | | | |
| MTBR tau-243 | 2.44 (0.63) | 4.47 (3.51) | 6.18 (2.71) | 9.17 (5.32) | 2.75 (0.89) |
| MTBR tau-299 | 0.39 (0.13) | 0.80 (0.45) | 1.14 (0.42) | 1.00 (0.49) | 0.45 (0.17) |
| MTBR tau-354 | 2.20 (0.41) | 2.73 (0.79) | 3.21 (0.77) | 2.73 (0.80) | 2.31 (0.49) |

Data are shown as mean (SD). AD: Alzheimer's disease. CI: cognitive impairment. CDR: Clinical Dementia Rating. PiB: Pittsburgh compound B. AV-45: florbetapir. AV-1451: flortaucipir. SUVR: standardized uptake value ratio. na: not available. Superscript numbers indicate the number of available measures within the group. Amyloid statuses in longitudinal and tau PET cohorts were determined historically from the results of the cross-sectional cohort and amyloid PET, respectively. The concentration of each MTBR tau isoform (MTBR tau-243, MTBR tau-299, and MTBR tau-354) was determined by mass spectrometry following to chemical extraction method in post-immunoprecipitated CSF samples.

TABLE 1B
Longitudinal and Tau PET cohorts

| | Longitudinal cohort (n = 28) | | Tau PET cohort (n = 35) | |
|---|---|---|---|---|
| Variable | Control | AD | Control | AD |
| n | 14 | 14 | 15 | 20 |
| Age | 74 (5) | 77 (6) | 75 (6) | 75 (6) |
| Gender (F/M) | 5/9 | 7/7 | 12/3 | 11/9 |
| CDR | 0-0.5 | 0-2 | 0-0.5 | 0-2 |
| CSF Aβ 42/40 | na | na | na | na |
| PiB SUVR | 1.11 (0.18) 13 | 2.47 (0.59) 11 | 1.10 (0.13) 10 | 2.37 (0.34) 14 |
| AV45 SUVR | 0.98 (0.19) 9 | 2.10 (0.46) 12 | 0.98 (0.26) 13 | 2.09 (0.49) 18 |
| Amyloid status | negative | positive | negative | positive |
| AV-1451 SUVR | na | na | 1.23 (0.14) | 1.75 (0.69) |
| CSF tau level (ng/mL) | | | | |
| MTBR tau-243 | 2.94 (0.87) | 7.09 (4.76) | 2.70 (0.67) | 6.82 (4.26) |
| MTBRtau-299 | 0.44 (0.23) | 1.18 (0.47) | 0.40 (0.21) | 1.07 (0.31) |
| MTBR tau-354 | 2.24 (0.48) | 3.61 (0.89) | 2.33 (0.64) | 3.43 (0.72) |

Data are shown as mean (SD). AD: Alzheimer's disease. CI: cognitive impairment. CDR: Clinical Dementia Rating. PiB: Pittsburgh compound B. AV-45: florbetapir. AV-1451: flortaucipir. SUVR: standardized uptake value ratio. na: not available. Superscript numbers indicate the number of available measures within the group. Amyloid statuses in longitudinal and tau PET cohorts were determined historically from the results of the cross-sectional cohort and amyloid PET, respectively. The concentration of each MTBR tau isoform (MTBR tau-243, MTBR tau-299, and MTBR tau-354) was determined by mass spectrometry following to chemical extraction method in post-immunoprecipitated CSF samples.

Brain tau analysis by MS: Frozen brain tissue samples were sliced using a cryostat at −20° C. and collected in tubes. The tissue (300-400 mg) was sonicated in ice-cold buffer containing 25 mM tris-hydrochloride (pH 7.4), 150 mM sodium chloride, 10 mM ethylenediaminetetraacetic acid, 10 mM ethylene glycol tetraacetic acid, phosphatase inhibitor cocktail, and protease inhibitor cocktail at a concentration of 0.3 mg/μL of brain tissue. The homogenate was clarified by centrifugation for 20 minutes at 11,000 g at 4° C. The supernatant (whole brain extract) was aliquoted into new tubes and kept at −80° C. until use. The whole brain extract was incubated with 1% sarkosyl for 60 minutes on ice, followed by ultra-centrifugation at 100,000 g at 4° C. for 60 minutes to obtain an insoluble pellet. The insoluble pellet was resuspended with 200 μL of PBS followed by sonication and the insoluble suspension was kept at −80° C. until use.

For soluble tau analysis, tau species in whole brain extract were immunoprecipitated with Tau1 and HJ8.5 antibodies. Immunoprecipitated soluble tau species were processed and digested as described previously (Sato et al., 2018).

For insoluble tau analysis, insoluble suspension (10 to 20 μL containing 2.5 μg of total protein) was mixed with 200 μL of lysis buffer (7 M urea, 2 M thio-urea, 3% 3-[(3-cholamidopropyl)dimethylammonio]-1-propanesulfonate, 1.5% n-octyl glucoside, 100 mM triethyl ammonium bicarbonate (TEABC)) followed by spiking with 5 μL of solution containing ¹⁵N Tau-441 (2N4R) Uniform Labeled (2 ng/μL, gift from Dr. Guy Lippens, Lille University, France) as an internal standard. Five μL of 500 mM dithiothreitol was added to the suspension, followed by sonication. The resulting solution was mixed with 15 μL of 500 mM iodoacetamide and incubated for 30 minutes at room temperature in the dark. Protein digestion was conducted using the filter-aided sample preparation method as previously reported (Roberts et al., 2020). Briefly, each prepared solution was loaded on a Nanosep 10K filter unit (PALL) and centrifuged. After washing the sample on the filter unit with 8 M urea in 100 mM TEABC solution, immobilized proteins were digested on the filter using 0.25 μg of endoproteinase Lys-C at 37° C. for 60 minutes. Then, the samples were further digested using 0.4 μg trypsin at 37° C. overnight.

The digested samples (soluble and insoluble tau species) were collected by centrifugation, then desalted by C18 TopTip (Glygen). In this purification process, 50 fmol each of AQUA internal-standard peptide for residues 354-369 (MTBR tau-354) and 354-368 (tau368) was spiked for the differential quantification. Before eluting samples, 3% hydrogen peroxide and 3% formic acid (FA) in water were added onto the beads, followed by overnight incubation at 4° C. to oxidize the peptides containing methionine. The eluent was lyophilized and resuspended in 27.5 μL of 2% acetonitrile and 0.1° A FA in water prior to MS analysis on nanoAcquity UPLC system (Waters) coupled to Orbitrap Fusion Tribrid or Orbitrap Tribrid Eclipse (Thermo Scientific) operating in parallel reaction monitoring (PRM) mode.

Sixteen brain tau peptides from both soluble and insoluble tau species were quantified by comparison with corresponding isotopomers signals from the ¹⁵N or AQUA internal standard (Table 2). Peptide-profile comparisons across brain samples were performed by normalizing each peptide amount by a mid-domain tau peptide (residue 181-190).

TABLE 2

| | Tau-441 | |
|---|---|---|
| | residues | |
| Peptide | (Abbreviated) | Sample matrix |
| QEFEVMEDHAGTYGLGDR (SEQ ID NO: 11) | 6-23 | Brain, CSF |
| DQGGYTMHQDQEGDTDAGLK (SEQ ID NO: 12) | 25-44 | Brain, CSF |
| ESPLQTPTEDGSEEPGSETSDAK | 45-67 | Brain, CSF |
| (SEQ ID NO: 13) | | |
| STPTAEDVTAPLVDEGAPGK (SEQ ID NO: 14) | 68-87 | Brain, CSF |
| QAAAQPHTEIPEGTTAEEAGIGDTPSLEDEAAGHV | 88-126 | CSF |
| TQAR (SEQ ID NO: 15) | | |
| IATPR (SEQ ID NO: 16) | 151-155 | Brain, CSF |
| TPPSSGEPPK (SEQ ID NO: 10) | 181-190 | Brain, CSF |
| SGYSSPGSPGTPGSR (SEQ ID NO: 17) | 195-209 | Brain, CSF |
| TPSLPTPPTR (SEQ ID NO: 18) | 212-221 | CSF |
| VAVVR (SEQ ID NO: 19) | 226-230 | Brain, CSF |
| LQTAPVPMPDLK (SEQ ID NO: 3) | 243-254 | Brain, CSF |
| | (MTBR tau-243) | |
| IGSTENLK (SEQ ID NO: 2) | 260-267 | Brain, CSF |
| VQIINK (SEQ ID NO: 4) | 275-280 | Brain, CSF |
| LDLSNVQSK (SEQ ID NO: 5) | 282-290 | CSF |
| HVPGGGSVQIVYKPVDLSK (SEQ ID NO: 6) | 299-317 | Brain, CSF |
| | (MTBR tau-299) | |
| IGSLDNITHVPGGGNK (SEQ ID NO: 7) | 354-369 | Brain, CSF |
| | (MTBR tau-354) | |
| IGSLDNITHVPGGGN (SEQ ID NO: 8) | 354-368 | Brain, CSF |
| | (tau368) | |
| TDHGAEIVYK (SEQ ID NO: 20) | 386-395 | Brain, CSF |
| SPVVSGDTSPR (SEQ ID NO: 21) | 396-406 | Brain, CSF |

Abbreviations:
microtubule binding region (MTBR),
cerebrospinal fluid (CSF)

CSF tau analysis by MS: CSF (455 μL) was mixed with 10 μL of solution containing ¹⁵N Tau-441 (2N4R) Uniform Labeled (100 pg/μL) as an internal standard. The tau species consisting primarily of N-terminal to mid-domain regions were immunoprecipitated with Tau1 and HJ8.5 antibodies. Immunoprecipitated tau species were processed and digested as described previously (Sato et al., 2018). Subsequently, 20 μL of ¹⁵N-tau internal standard (100 pg/μL) was spiked into the post-immunoprecipitated CSF. Then, tau was chemically extracted as previously reported (Barthélemy et al., 2016b) with some modifications. Highly abundant CSF proteins were precipitated using 25 μL of perchloric acid. After mixing and incubation on ice for 15 minutes, the mixture was centrifuged at 20,000 g for 15 minutes at 4° C., and the supernatant was further purified using the Oasis HLB 96-well μElution Plate (Waters) according to the following steps. The plate was washed once with 300 μL of methanol and equilibrated once with 500 μL of 0.1% FA in water. The supernatant was added to the Oasis HLB 96-well μElution Plate and adsorbed to the solid phase. Then, the solid phase was washed once with 500 μL of 0.1% FA in water. Elution buffer (100 μL; 35% acetonitrile and 0.1% FA in water) was added, and the eluent was dried by Speed-vac. Dried sample was dissolved by 50 μL of trypsin solution (10 ng/μL) in 50 mM TEABC and incubated at 37° C. for 20 hours.

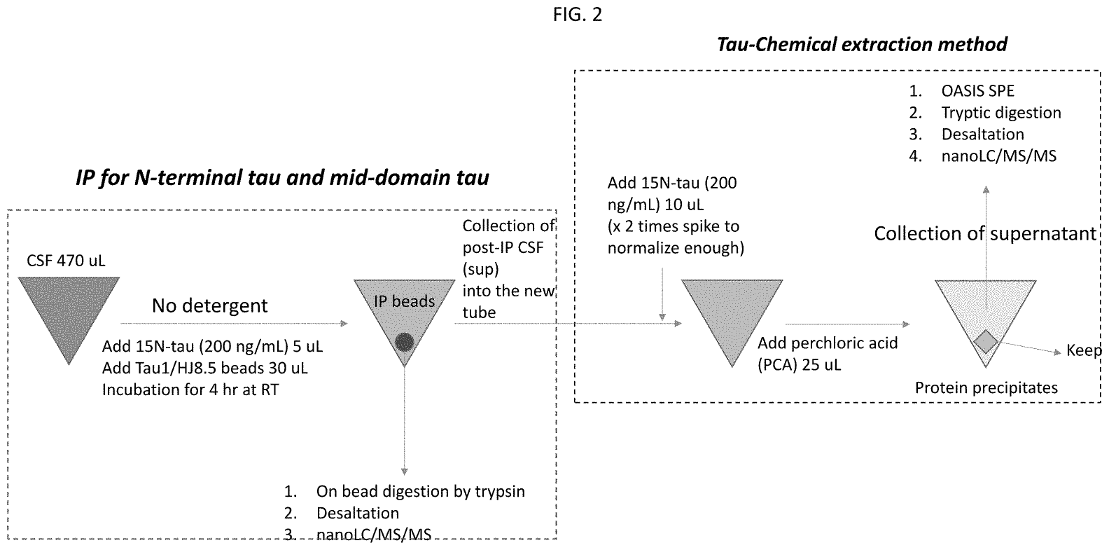

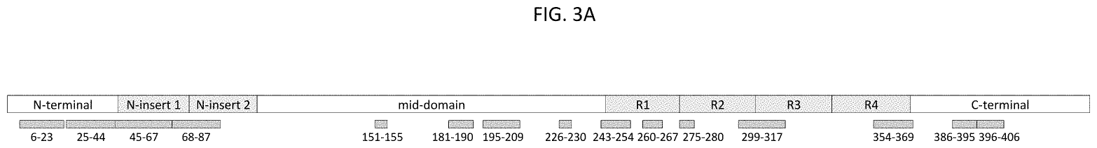

After incubation for both immunoprecipitated and chemically extracted samples, each tryptic digest was purified by solid phase extraction on C18 TopTip. In this purification process, 5 fmol each of AQUA internal-standard peptide for residues 354-369 (MTBR tau-354) and 354-368 (tau368) was spiked for the differential quantification. Before eluting samples, 3% hydrogen peroxide and 3% FA in water were added to the beads, followed by overnight incubation at 4° C. to oxidize the peptides containing methionine. The eluent was lyophilized and resuspended in 27.5 μL of 2% acetonitrile and 0.1% FA in water prior to MS analysis on nanoAcquity UPLC system coupled to Orbitrap Fusion Lumos Tribrid or Orbitrap Tribrid Eclipse mass spectrometer (Thermo Scientific) operating in PRM mode. Nineteen CSF tau peptides were quantified (Table 4). The schematic procedure of CSF tau analysis is described in FIG. 2.

Statistical analysis: Differences in biomarker values were assessed with one-way ANOVAs, unless otherwise specified. A two-sided p<0.05 was considered statistically significant and corrected for multiple comparisons using Benjamini-Hochberg false discovery rate (FDR) method with FDR set at 5% (Benjamini and Hochberg, 1995). Spearman correlations were used to assess associations between tau biomarkers and cognitive testing measures and tau PET SUVR.

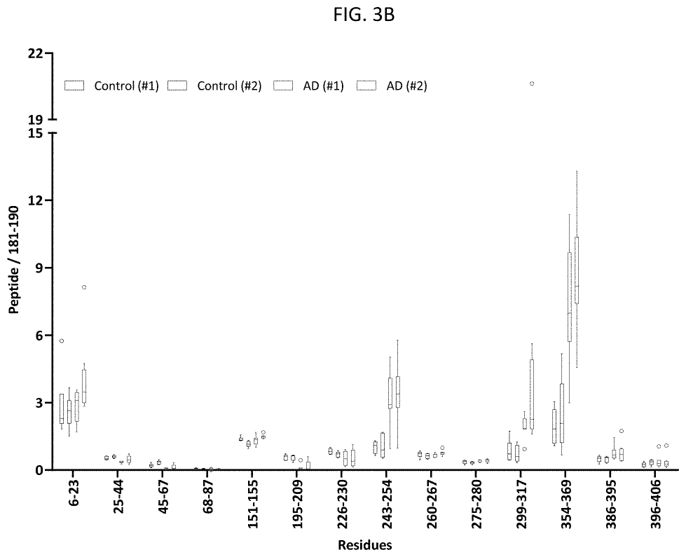

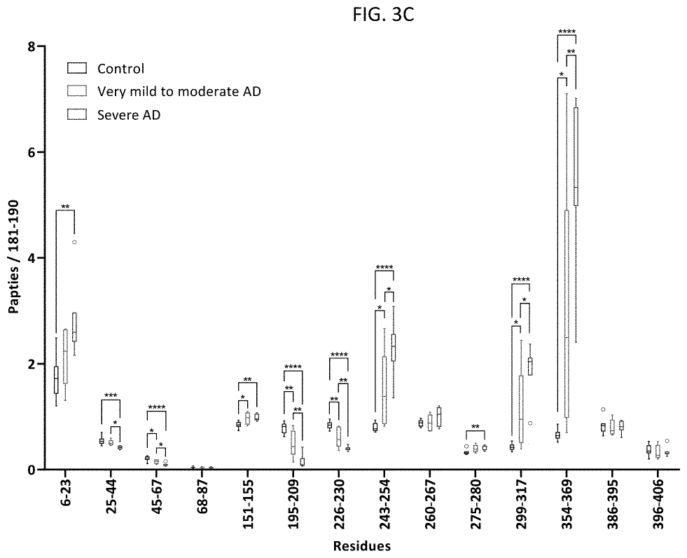

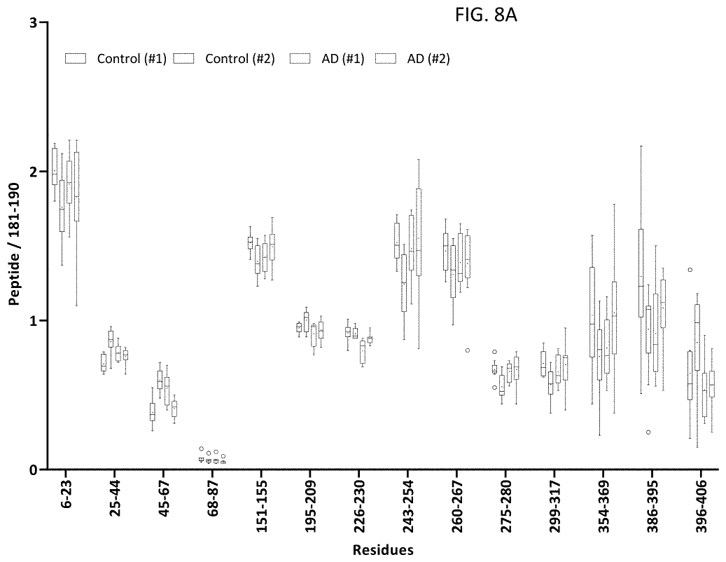

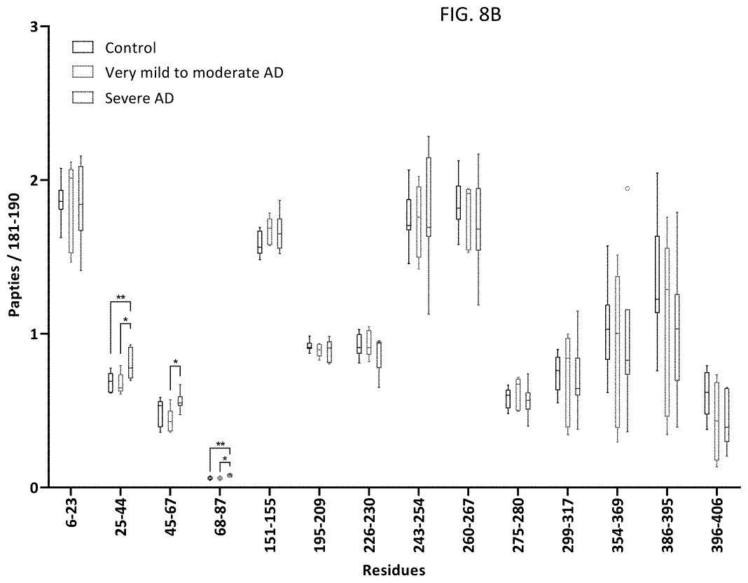

Results—Enrichment profiling of tau species in Alzheimer's disease brain: It was hypothesized that tau aggregation in Alzheimer's disease brain would be reflected in tau profiles in CSF. Therefore, tau profiles in insoluble extracts from Alzheimer's disease and control brains were first analyzed (FIG. 3B: discovery cohort) to later compare with CSF tau profiles. It was determined that the species containing residues 299-317 (MTBR tau-299) and 354-369 (MTBR tau-354), located between R2 and R3 domains and within the R4 domain, respectively, were more enriched in the insoluble extract from Alzheimer's disease brain than control by 3-4 fold. The upstream region of MTBR containing residues 243-254 (MTBR tau-243) was also about three times greater in Alzheimer's disease brain as compared to the control, while species containing residues 260-267 and 275-280 located within R1 and R2 domains, respectively, did not differ between Alzheimer's disease and control tissues. No other regions of tau were enriched in Alzheimer's disease brain compared to controls. Notably, the species containing residue 195-209 within the mid-domain was particularly lower in Alzheimer's disease brain compared to the control, likely the result of extensive hyper-phosphorylation occurring on residues 199, 202, 205, and 208 in insoluble tau aggregates (Malia et al., 2016). Of note, no change was observed for the identified MTBR tau species in soluble tau (whole brain extract) between control and Alzheimer's disease (FIG. 8A). These results were reproduced in brain samples from control (amyloid-negative, n=8), very mild to moderate Alzheimer's disease (amyloid-positive, CDR=0.5-2, n=5) and severe Alzheimer's disease (amyloid-positive, CDR=3, n=7) participants (FIG. 3C and FIG. 8B: validation cohort), which suggested MTBR tau-243, 299, and 354 species were specifically enriched in insoluble tau aggregates over the stages of disease progression.

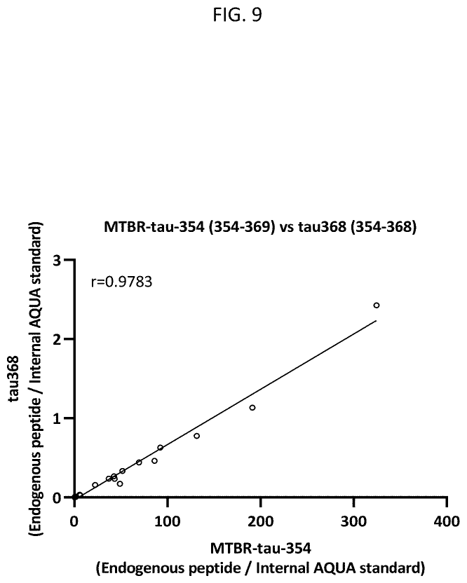

Next, the recently reported truncated tau368 (residue 354-368) species generated by asparagine endopeptidase (Zhang et al., 2014; Blennow et al., 2020) was examined against its paired non-truncated species, MTBR tau-354, and quantified both species in brain insoluble extracts (FIG. 9). A high correlation between tau368 and MTBR tau-354 was found (r=0.9783), suggesting that truncation at residue 368 occurs at the same rate in different stages of brain pathology.

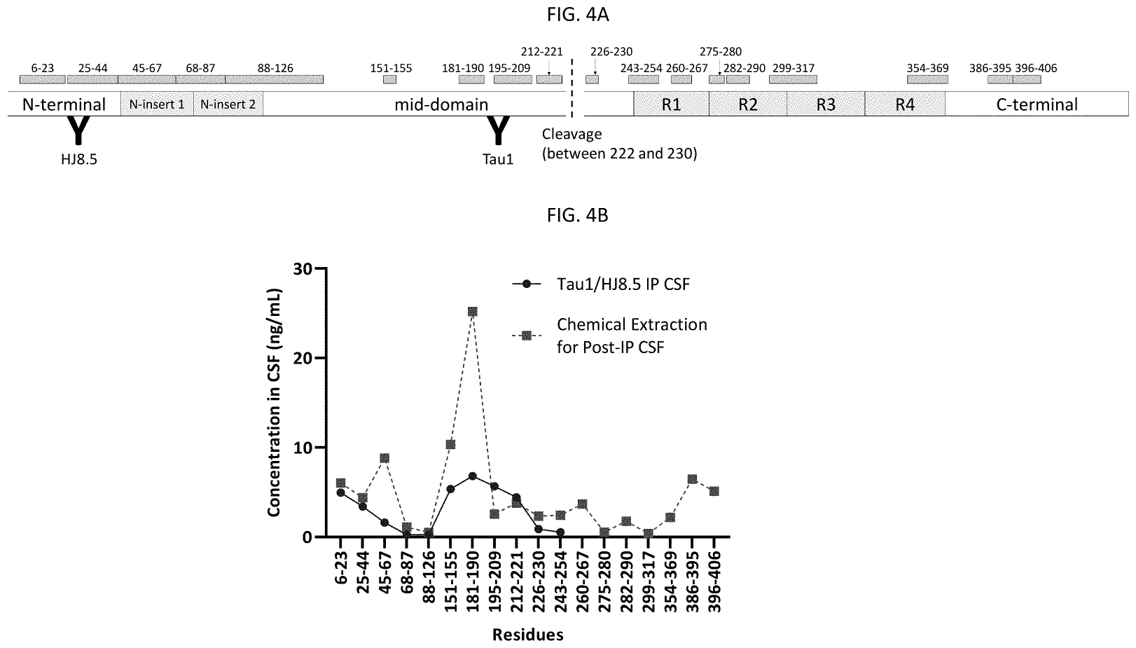

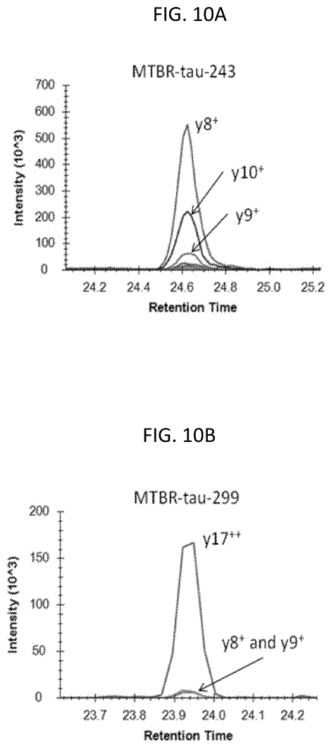

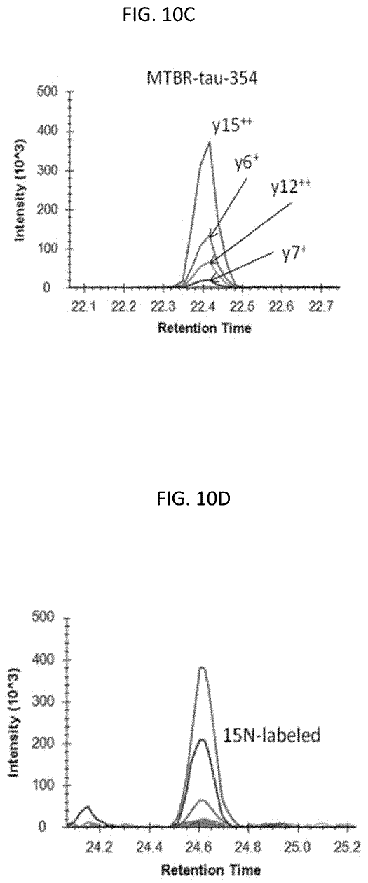

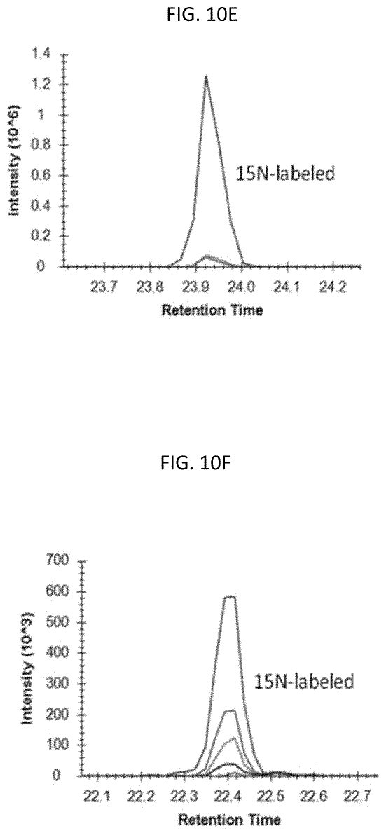

Results—Quantification of MTBR tau in CSF: To determine whether the enrichment of MTBR tau in Alzheimer's disease brain aggregates are related to levels of soluble tau species in the CSF, a method was developed to analyze MTBR tau in CSF. The method utilizes tau chemical extraction in post-immunoprecipitated (Tau1/HJ8.5) CSF followed by MS analysis (FIG. 2). This method provided sufficient recovery for quantifying MTBR peptides (FIG. 10). Tau peptide abundance recovered by Tau1/HJ8.5 immunoprecipitation method before chemical extraction was dramatically decreased after residue 222 (Sato et al., 2018). In contrast, the concentrations of MTBR tau species quantified by the PostIP-CX method were relatively low compared to the N-terminus to mid domain regions but still comparable to the other regions of tau by immunoprecipitation (FIG. 4). CSF concentrations from normal control participants (calculated as total values from immunoprecipitation and chemical extraction methods) ranged from 8.2 to 32.0 ng/mL for mid-domain species (residues 151-155, 181-190, 195-209, and 212-221), 0.4 to 3.7 ng/mL for MTBR tau species (residues 243-254, 260-267, 275-280, 282-290, 299-317, and 354-369), and 6.5 and 5.1 ng/mL for non-MTBR C-terminal tau species (residues 386-395 and 396-406). The CSF concentrations of C-terminal-containing truncated tau species were in a similar range as those containing the mid-domain (residues 195-209 and 212-221), suggesting the C-terminal side of tau is also truncated in neuronal cells and secreted extracellularly in the same manner as N-terminus to mid-domain tau (Sato et al., 2018).

Results—CSF MTBR tau in an Alzheimer's disease cross-sectional cohort: To determine whether MTBR-containing species present in the extracellular space reflect Alzheimer's disease-related changes, CSF was analyzed from a cross-sectional cohort of amyloid-negative and amyloid-positive participants at different clinical stages: amyloid-negative CDR=0 (control, n=30), amyloid-positive CDR=0 (preclinical AD, n=18), amyloid-positive CDR=0.5 (very mild AD, n=28), amyloid-positive CDR≥1 (mild-moderate AD, n=12), and amyloid-negative CDR≥0.5 (non-AD cognitive impairment, n=12).

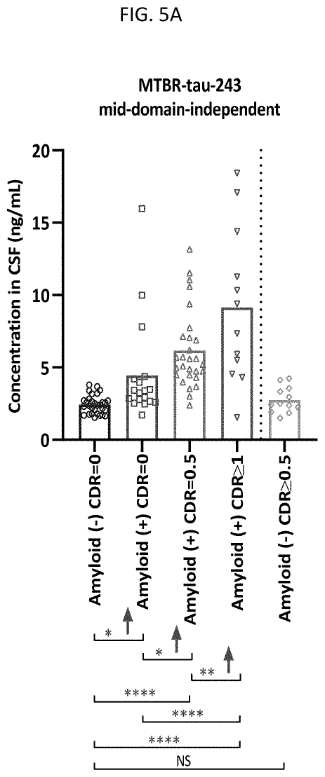

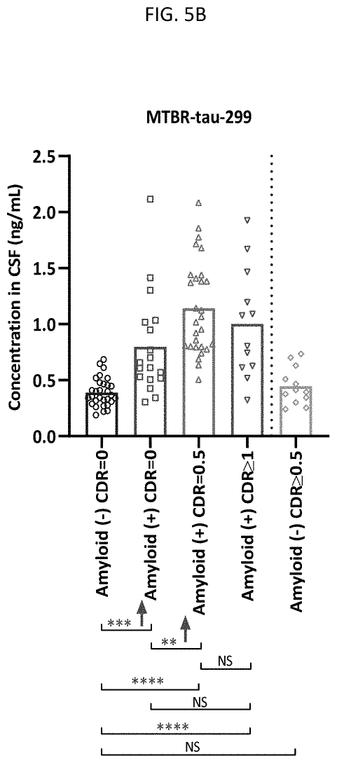

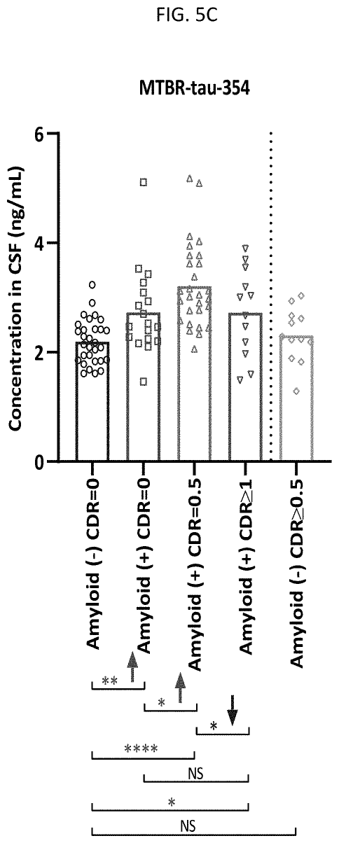

First, CSF levels of three MTBR tau species specifically enriched in Alzheimer's disease brain (MTBR tau-243, MTBR tau-299, and MTBR tau-354) were investigated (FIG. 5). All three species were present in both Alzheimer's disease and control CSF and levels were greater in the amyloid-positive groups even for the asymptomatic stage (CDR=0) when compared to the control group (MTBR tau-243 p=0.0170, MTBR tau-299 p=0.0002, and MTBR tau-354 p=0.0076). Remarkably, these species had distinct characteristics in CSF after clinical disease onset. MTBR tau-299 levels were 204% higher in preclinical AD compared to controls but saturated between very mild AD (CDR=0.5) and mild-moderate AD (CDR≥1) (p=0.2541), while MTBR tau-354 levels were significantly lower in samples collected post-symptom onset (p=0.0345). In contrast, MTBR tau-243 levels were incrementally higher across all disease stages including after symptom onset (p=0.0025). These results suggest that the regional specificity even within the MTBR tau species can distinguish among different Alzheimer's disease stages and that MTBR tau-243 is a good Alzheimer's disease stage-specific marker.

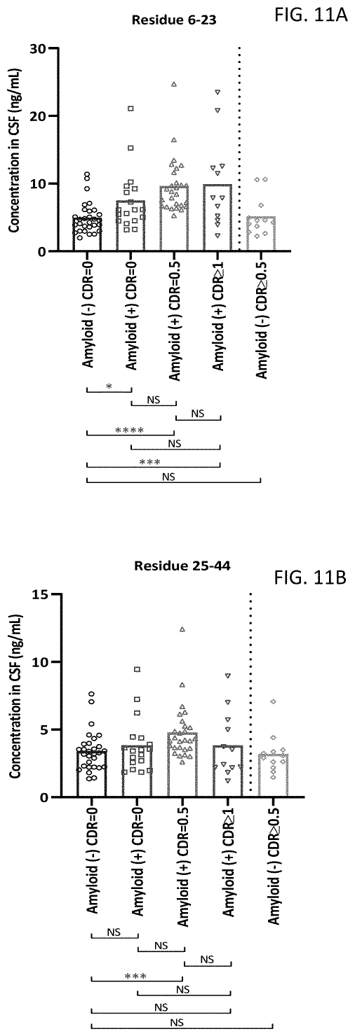

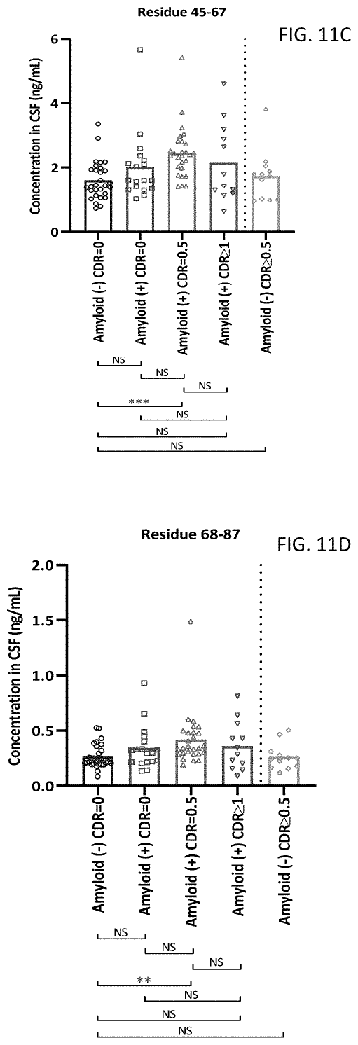

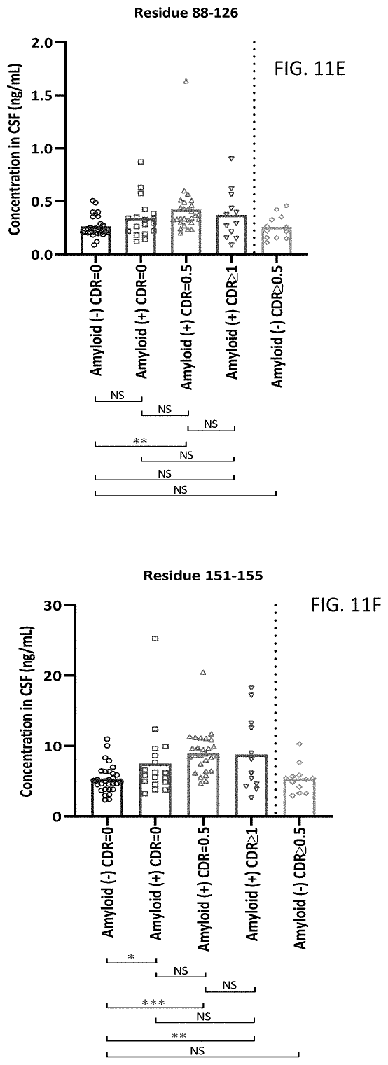

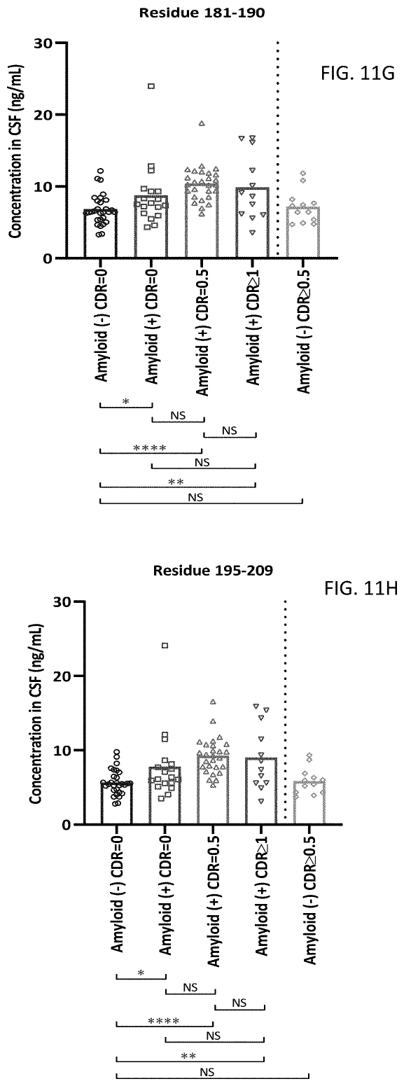

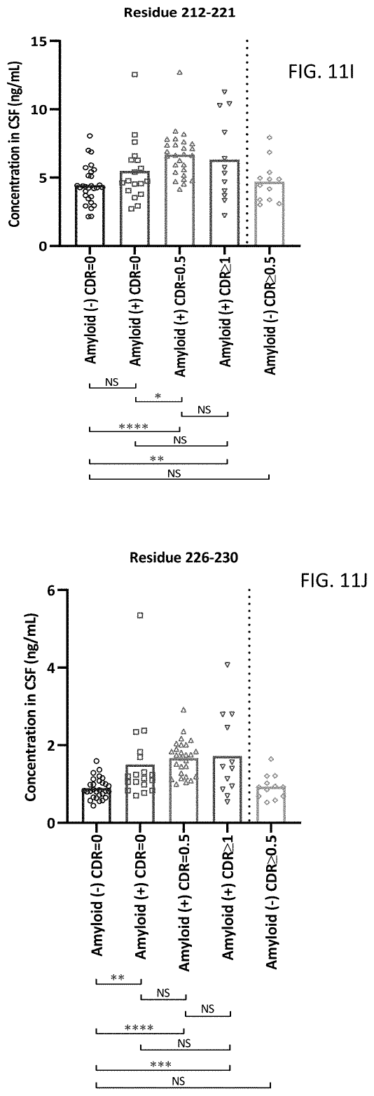

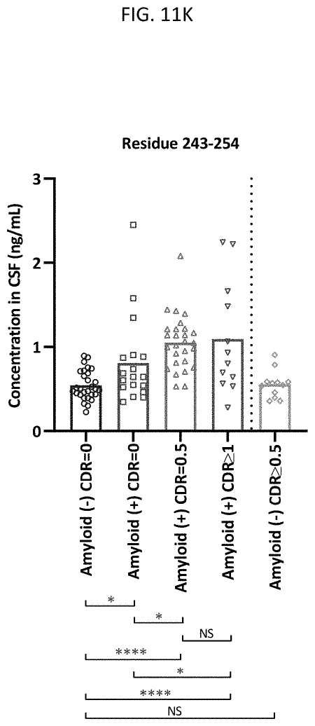

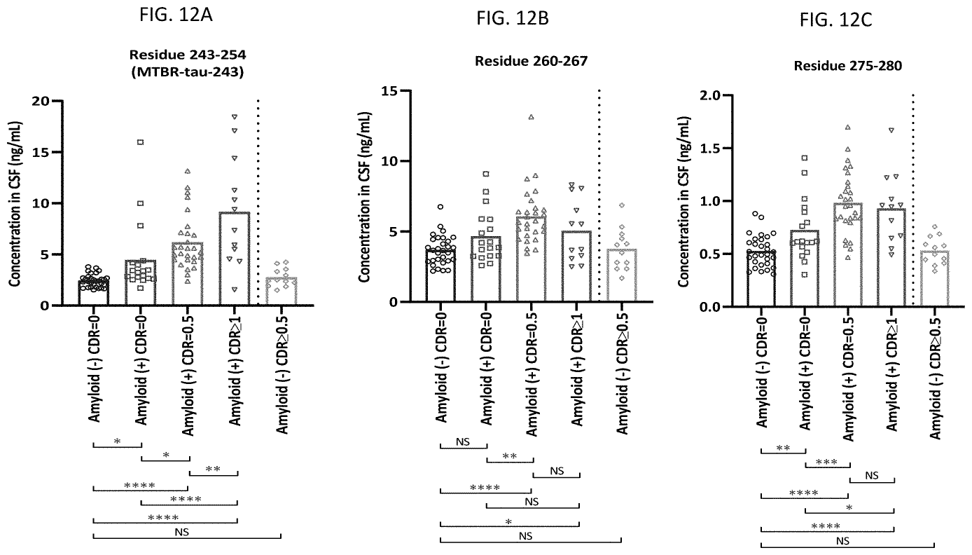

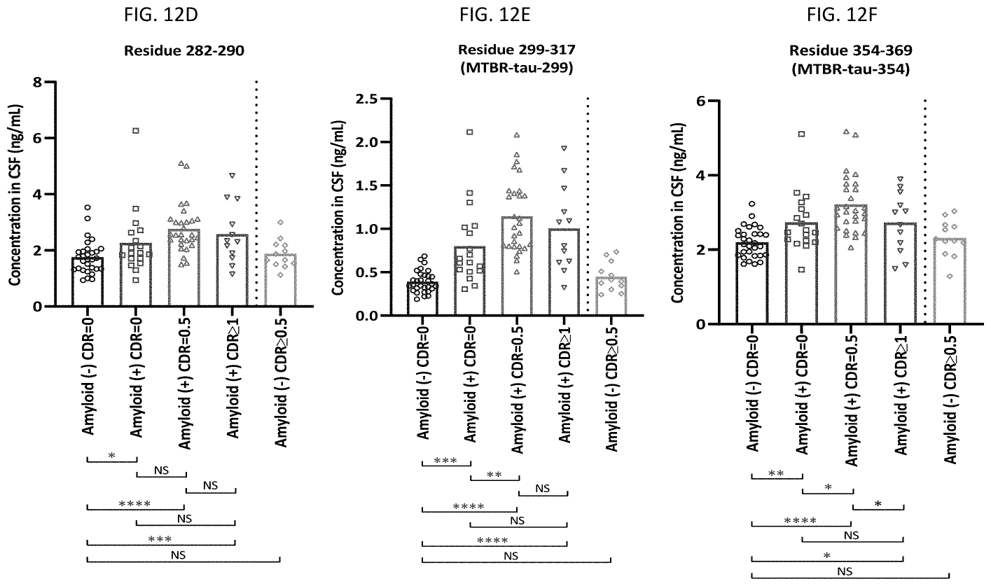

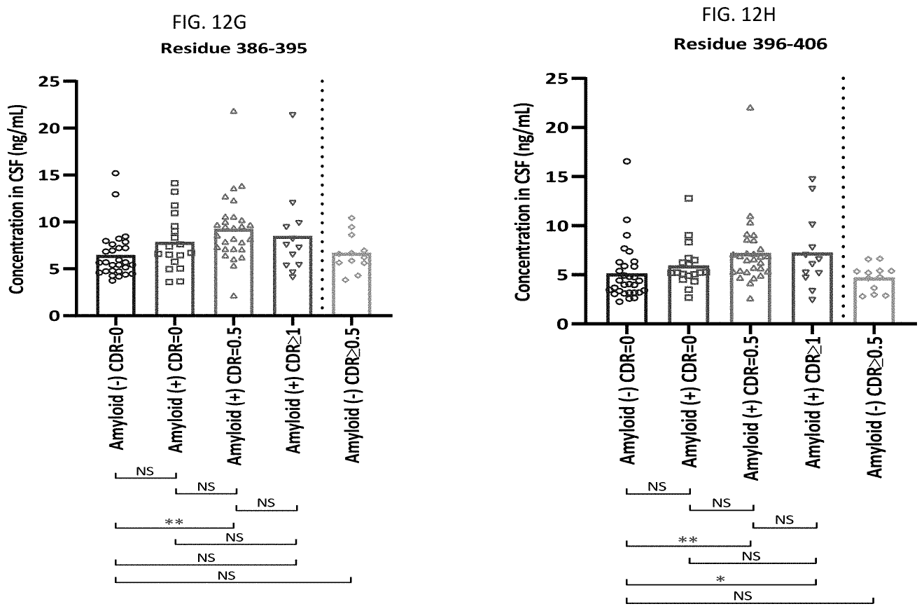

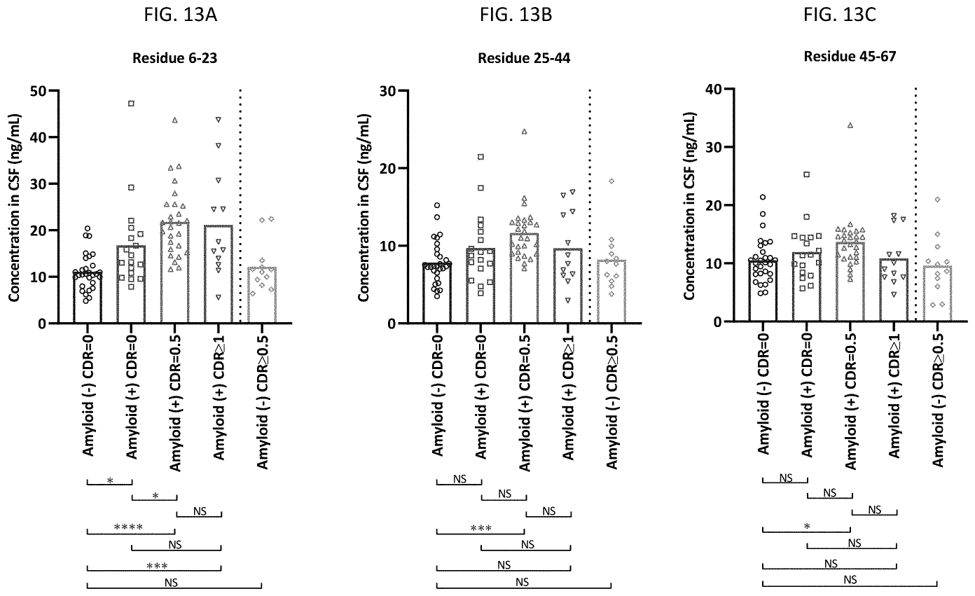

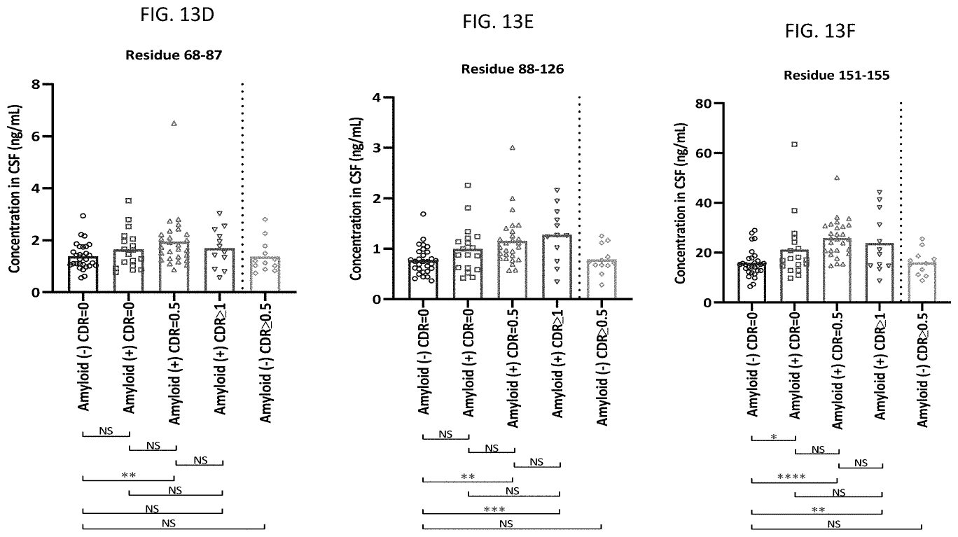

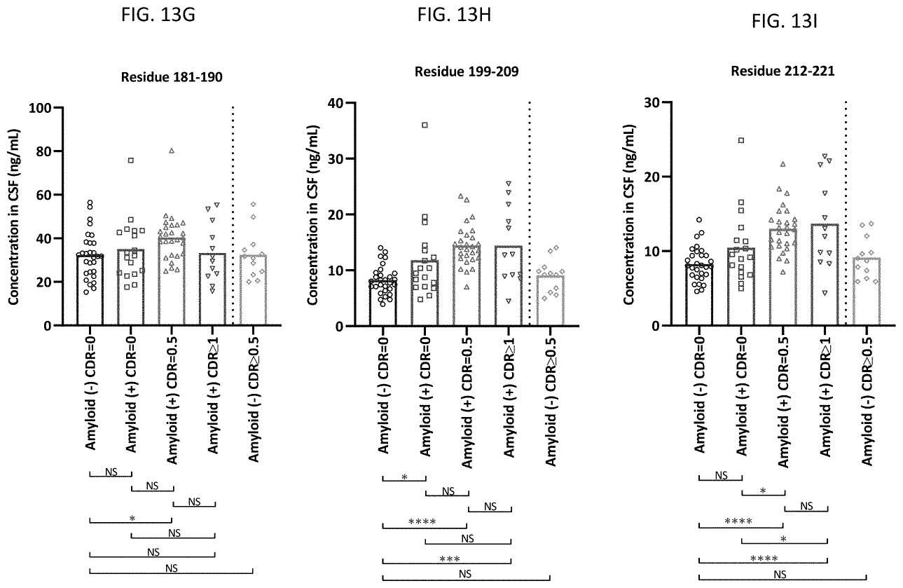

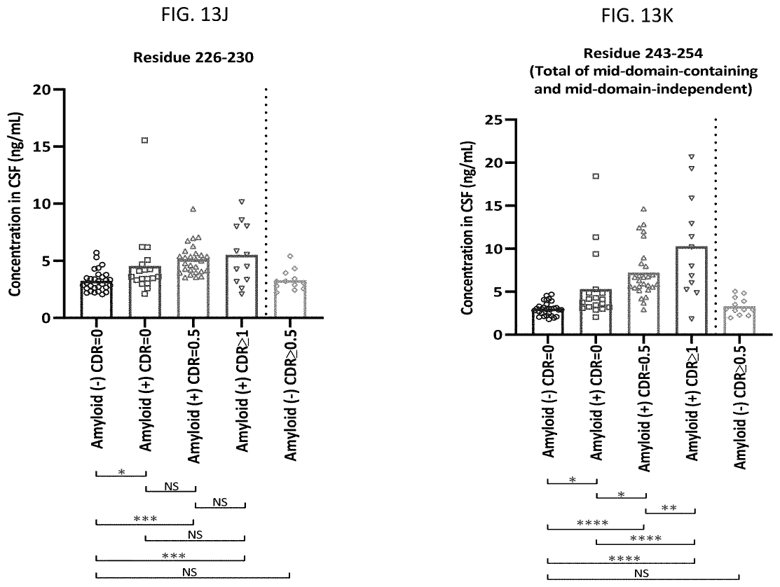

Next investigated was whether CSF MTBR tau species provide enhanced sensitivity and specificity in staging Alzheimer's disease when compared to tau species containing other regions. Multiple species containing N-terminal, mid-domain, MTBR, and C-terminal domains were quantified by region-specific methods (FIG. 11, FIG. 12, FIG. 13). N-terminal and mid-domain species were quantified by immunoprecipitation (IP method) as well as chemical extraction for post-immunoprecipitated CSF (PostIP-CX method), while the MTBR to C-terminal species were quantified only by the chemical extraction method for post-immunoprecipitated CSF (PostIP-CX method) because quantifiable signals were not obtained by immunoprecipitation. Levels of species containing N-terminal domains quantified by the immunoprecipitation method were not different between the control and asymptomatic stage (except for residue 6-23, p=0.0362) or other neighboring disease stages. The mid-domain species levels by the immunoprecipitation method were significantly greater in the asymptomatic amyloid stage than in controls (except for residue 212-221, p=0.0762) but the effect size was relatively modest (123%-168% vs. control) compared to the MTBR tau species (e.g., MTBR tau-299 levels were >200% greater at the preclinical AD stage than control) and did not differ across later disease stages. Regardless of the extraction method, MTBR tau-243, 299, and 354 species showed greater differences between control and disease stages compared to N-terminal to mid-domains species (residues 6-23 to 226-230, FIG. 13). Profiles from the other species containing MTBR to C-terminal domains (residues 260-267, 275-280, 282-290, 386-395, and 396-406) were similar to the mid-domain species and were not specific for the stage of Alzheimer's disease clinical dementia.

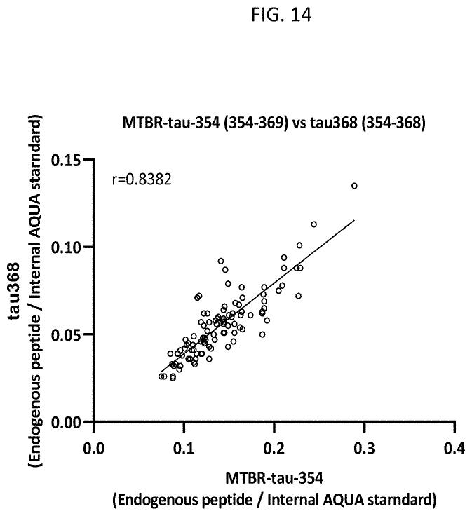

In summary, the three representative species containing MTBR (MTBR tau-243, MTBR tau-299, and MTBR tau-354) that were enriched in Alzheimer's disease brain (FIG. 3) had similar characteristics in CSF with MTBR tau-243 exhibiting the greatest specificity to Alzheimer's disease dementia stage. Of note, a high correlation was observed between the tau368 truncated form and MTBR tau-354 non-truncated form in CSF (r=0.8382) (FIG. 14). The only species that could reliably distinguish the clinical stages of Alzheimer's disease was MTBR tau-243.

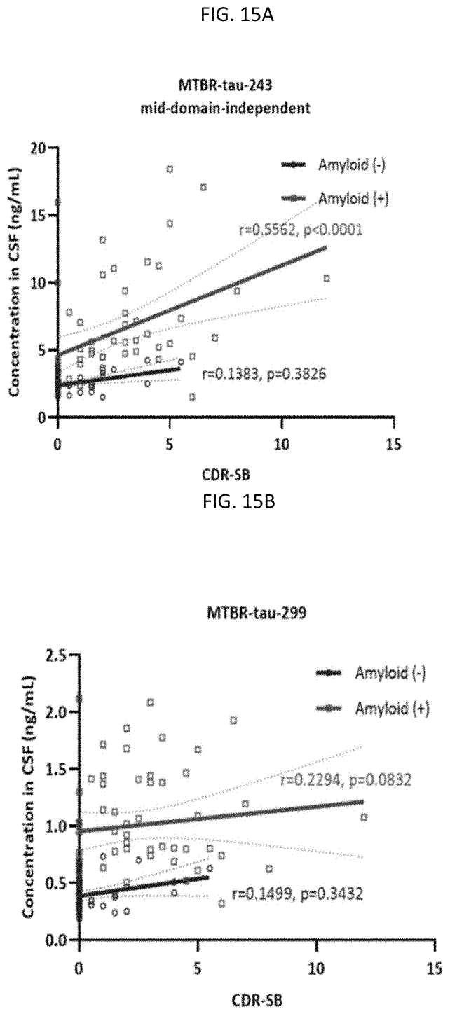

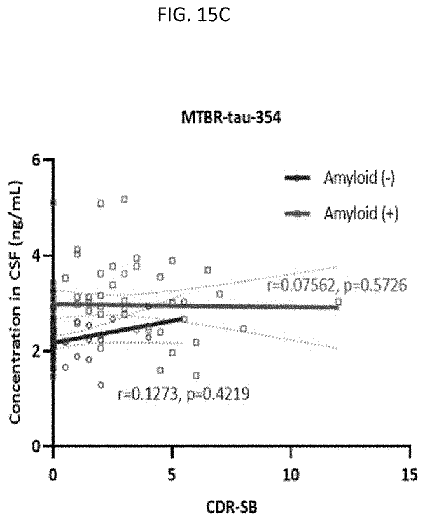

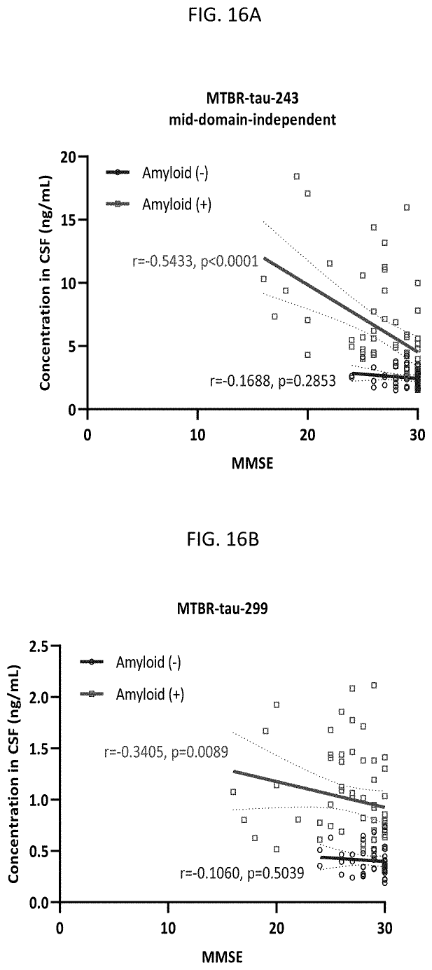

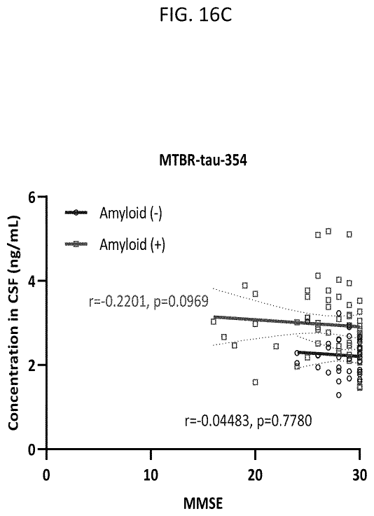

Results—Mid-domain-independent MTBR tau-243 as a specific biomarker to stage Alzheimer's disease: The incrementally greater levels of the MTBR tau-243 species across Alzheimer's disease clinical dementia stages suggest it may be a reliable predictor of disease progression. Next investigated was which MTBR tau species (MTBR tau-243, MTBR tau-299, and MTBR tau-354) had the highest correlations with results of cognitive tests such as CDR-sum of boxes (CDR-SB) and the Mini-Mental State Exam (MMSE). It was found that the mid-domain-independent MTBR tau-243 species in the amyloid-positive group was highly correlated with both CDR-SB and MMSE (r=0.5562, p<0.0001 and r=−0.5433, p<0.0001, respectively) (FIG. 15 and FIG. 16). Other species levels had much lower or no significant correlations with the cognitive testing (Table 3), which suggests that CSF MTBR tau-243 specifically differentiates clinical stage and global disease progression from the asymptomatic stage through advancing clinical stages of Alzheimer's disease.

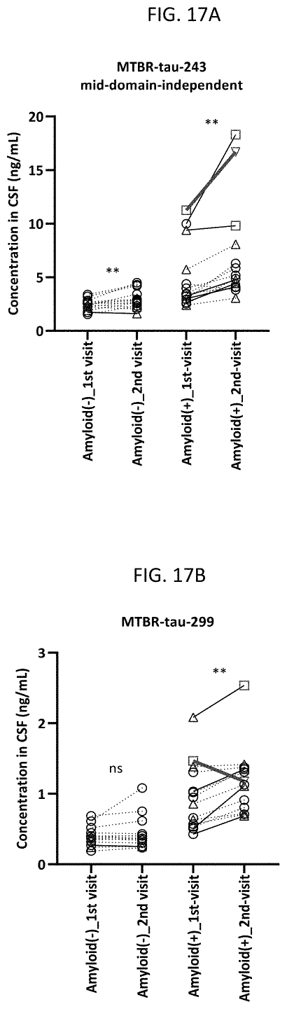

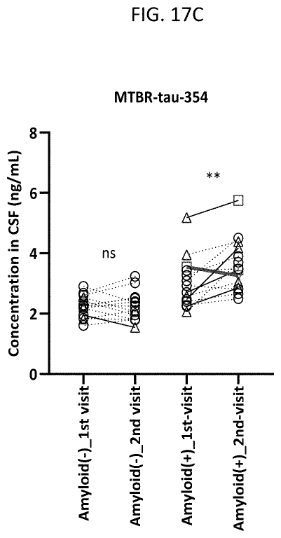

Results—CSF MTBR tau in an Alzheimer's disease longitudinal cohort: From the cross-sectional cohort, a subset of participants (n=28) were followed for two to nine years to measure the longitudinal trajectory of MTBR tau in CSF (Table 4). MTBR tau species enriched in Alzheimer's disease brain (MTBR tau-243, MTBR tau-299, and MTBR tau-354) were significantly increased over time in the amyloid-positive group (p<0.01 by two-tailed paired t-test between 1^{st} and 2^{nd} visits) but not the amyloid-negative group, except for MTBR tau-243 (FIG. 17). The amyloid-negative group also showed slight longitudinal increases of MTBR tau-243 but lower than observed for the amyloid-positive group (means of differences=0.4926 and 2.208 in amyloid-negative and positive groups, respectively).

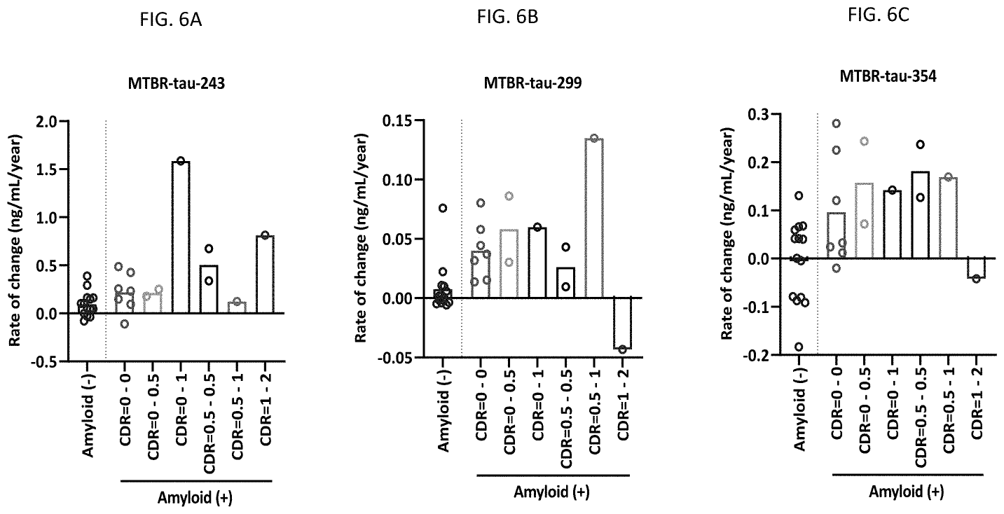

FIG. 6 shows the longitudinal change-rates of the MTBR tau species concentrations in individual participants. Notably, one participant (participant A) with the highest CDR after disease onset (changed from CDR=1 to 2 in seven years) showed specific trajectory profiles for each MTBR tau species. MTBR tau-243 continuously increased even from mild AD (CDR=1) to moderate AD (CDR=2), while MTBR tau-299 and MTBR tau-354 showed a decrease in this participant's CSF after mild AD. Other participants in the amyloid-positive group were classified as preclinical AD or very mild AD (CDR=0 or 0.5, respectively) at the 1^{st} visit, and the increasing trend for each species level was seen for most of the participants, which supports the findings from the cross-sectional cohort.

TABLE 4

| | | | | | MTBR tau-243 | | MTBR tau-299 | | MTBR tau-354 | |
|---|---|---|---|---|---|---|---|---|---|---|
| | | Visit | CDR | | (ng/mL) | | (ng/mL) | | (ng/mL) | |
| Participant | Amyloid | interval | Visit | Visit | Visit | Visit | Visit | Visit | Visit | Visit |
| ID | status | (year) | 1 | 2 | 1 | 2 | 1 | 2 | 1 | 2 |
| 1 | Negative | 7.0 | 0.5 | 0 | 1.916 | 2.645 | 0.241 | 0.254 | 1.824 | 2.106 |
| 2 | Negative | 4.9 | 0 | 0 | 2.220 | 2.043 | 0.317 | 0.300 | 2.179 | 1.794 |
| 3 | Negative | 4.5 | 0.5 | 0 | 2.245 | 2.728 | 0.395 | 0.404 | 2.236 | 2.535 |
| 4 | Negative | 8.0 | 0 | 0 | 2.569 | 2.560 | 0.446 | 0.430 | 1.841 | 1.805 |
| 5 | Negative | 5.2 | 0 | 0 | 2.705 | 2.946 | 0.366 | 0.341 | 2.252 | 2.256 |
| 6 | Negative | 5.3 | 0 | 0 | 2.588 | 2.817 | 0.399 | 0.403 | 2.289 | 2.506 |
| 7 | Negative | 6.2 | 0 | 0 | 3.386 | 4.306 | 0.612 | 1.080 | 2.610 | 3.026 |
| 8 | Negative | 6.2 | 0 | 0 | 3.205 | 4.200 | 0.685 | 0.752 | 2.904 | 2.364 |
| 9 | Negative | 5.7 | 0 | 0 | 2.550 | 2.866 | 0.399 | 0.366 | 2.419 | 1.955 |
| 10 | Negative | 4.7 | 0 | 0 | 1.545 | 2.322 | 0.188 | 0.235 | 1.612 | 1.809 |
| 11 | Negative | 4.3 | 0 | 0 | 2.822 | 4.494 | 0.519 | 0.616 | 2.676 | 3.239 |
| 12 | Negative | 4.4 | 0 | 0.5 | 1.729 | 1.604 | 0.269 | 0.252 | 1.948 | 1.546 |
| 13 | Negative | 3.6 | 0.5 | 0 | 2.372 | 3.421 | 0.345 | 0.365 | 2.187 | 2.400 |
| 14 | Negative | 2.6 | 0.5 | 0.5 | 2.432 | 2.229 | 0.376 | 0.372 | 2.539 | 2.062 |
| 15 | Positive | 5.2 | 0 | 1 | 9.992 | 18.307 | 1.034 | 1.347 | 2.704 | 3.449 |
| 16 | Positive | 8.6 | 0 | 0.5 | 3.234 | 4.752 | 0.425 | 0.685 | 2.241 | 2.856 |
| 17 | Positive | 6.7 | 1 | 2 | 11.253 | 16.698 | 1.466 | 1.177 | 3.546 | 3.265 |
| (participant | | | | | | | | | | |
| A) | | | | | | | | | | |
| 18 | Positive | 3.5 | 0.5 | 0.5 | 5.722 | 8.071 | 1.383 | 1.416 | 3.948 | 4.390 |
| 19 | Positive | 7.0 | 0 | 0.5 | 2.665 | 4.417 | 0.505 | 1.109 | 2.469 | 4.180 |
| 20 | Positive | 6.1 | 0 | 0 | 3.470 | 4.055 | 0.530 | 0.803 | 2.930 | 2.809 |
| 21 | Positive | 9.1 | 0 | 0 | 2.529 | 4.852 | 0.570 | 0.708 | 2.278 | 2.497 |
| 22 | Positive | 7.9 | 0 | 0 | 2.926 | 6.297 | 0.658 | 0.910 | 2.404 | 2.663 |
| 23 | Positive | 5.0 | 0 | 0 | 3.424 | 5.866 | 1.018 | 1.309 | 3.093 | 4.504 |
| 24 | Positive | 7.3 | 0.5 | 0 | 3.002 | 4.092 | 0.857 | 1.128 | 2.061 | 3.704 |
| 25 | Positive | 5.2 | 0 | 0 | 3.969 | 5.171 | 1.303 | 1.375 | 3.270 | 3.895 |
| 26 | Positive | 5.0 | 0 | 0 | 4.380 | 3.829 | 0.949 | 1.350 | 3.427 | 3.486 |
| 27 | Positive | 3.3 | 0.5 | 1 | 9.399 | 9.809 | 2.084 | 2.535 | 5.182 | 5.748 |
| 28 | Positive | 2.0 | 0.5 | 0.5 | 2.397 | 3.059 | 0.634 | 0.719 | 2.581 | 3.045 |

Abbreviations: Clinical Dementia Rating (CDR)

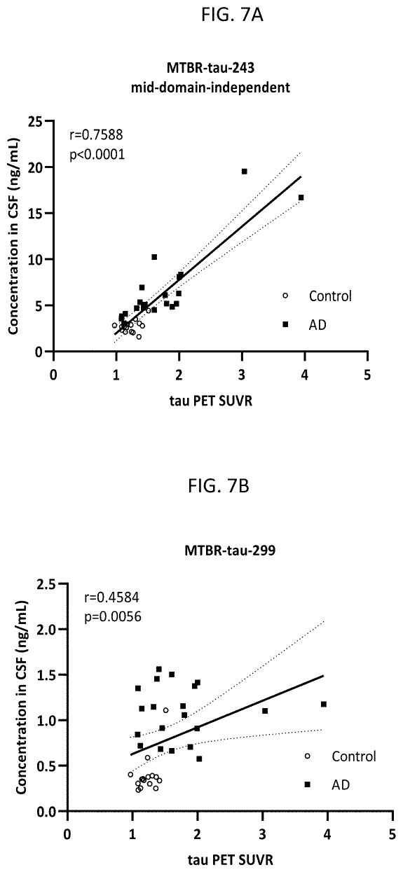

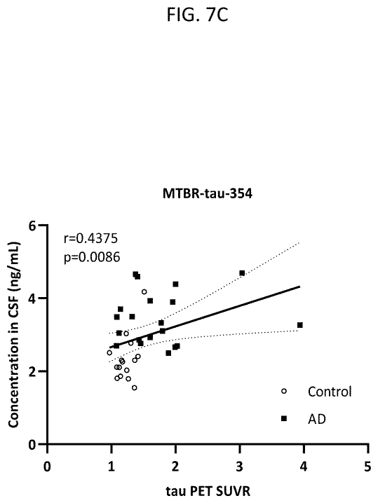

Results—Correlation with tau PET imaging: Tau pathology as measured by tau PET scans correlates strongly with cognitive decline and clinical stage of Alzheimer's disease (Arriagada et al., 1992; Johnson et al., 2016; Ossenkoppele et al., 2016; Bejanin et al., 2017; Jack et al., 2018; Gordon et al., 2019). Next investigated was whether MTBR tau in CSF was correlated with brain tau pathology measured by tau PET (FIG. 7). Mid-domain-independent MTBR tau-243 significantly correlated with tau PET SUVR (r=0.7588, p<0.0001), while MTBR tau-299 and MTBR tau-354 were much less correlated (r=0.4584, p=0.0056 and r=0.4375, p=0.0086, respectively). Tau species containing residue 226-230 also showed high correlation with tau PET SUVR (r=0.6248, p<0.0001, Table 5), but lower than observed for MTBR tau-243. This suggests that CSF MTBR tau-243 and the surrounding region may be surrogate biomarkers of tau aggregation in the brain. The ability to specifically and quantitatively track tau pathology in the brain is a much needed biomarker for Alzheimer's disease clinical studies.

TABLE 5
Correlations between each CSF tau species and tau PET
SUVR: mid-domain-independent MTBR tau-243 correlates
with tau pathology better than the other tau species

| Residue (Abbreviated) | Preparation method | r | p-value |
|---|---|---|---|
| 6-23 | IP | 0.5361 | 0.0011 |
| 25-44 | IP | 0.4411 | 0.0090 |
| 45-67 | IP | 0.5276 | 0.0013 |
| 68-87 | IP | 0.4700 | 0.0050 |
| 88-126 | IP | 0.3928 | 0.0215 |
| 151-155 | IP | 0.5501 | 0.0006 |
| 181-190 | IP | 0.5315 | 0.0010 |
| 195-209 | IP | 0.5471 | 0.0007 |
| 212-221 | IP | 0.4779 | 0.0037 |
| 226-230 | IP | 0.6248 | <0.0001 |
| 243-254 | IP | 0.6346 | <0.0001 |
| 243-254 (MTBR tau-243) | PostIP-CX | 0.7588 | <0.0001 |
| 260-267 | PostIP-CX | 0.3787 | 0.0249 |
| 275-280 | PostIP-CX | 0.5263 | 0.0012 |
| 282-290 | PostIP-CX | 0.5003 | 0.0022 |
| 299-317 (MTBR tau-299) | PostIP-CX | 0.4584 | 0.0056 |
| 354-369 (MTBR tau-354) | PostIP-CX | 0.4375 | 0.0086 |
| 386-395 | PostIP-CX | 0.4139 | 0.0185 |
| 396-406 | PostIP-CX | 0.3843 | 0.0248 |

Discussion: MTBR regions of tau have been investigated primarily in brain aggregates but not extensively in CSF. In this study, using a sensitive and antibody-independent method to analyze CSF tau, the presence and quantification of MTBR regions of tau in CSF samples from human participants was shown. Past studies utilizing antibody-dependent assays (Meredith et al., 2013; Sato et al., 2018) may have failed to detect MTBR-containing tau species in CSF due to assay limitations including antibody specificity or sensitivity, or the ability to recover potential conformations adopted by MTBR species in CSF. Alternatively, MTBR tau may be truncated by various proteases, generating fragments that are not detected in conventional immunoassays or immunoprecipitation followed by MS assays (Gamblin et al., 2003; Cotman et al., 2005; Zhang et al., 2014; Zhao et al., 2016; Chen et al., 2018; Quinn et al., 2018). In this study, surprisingly robust concentrations of MTBR tau species were measured, at about 1% to 10% compared to the mid-domain tau species, by using a PostIP-CX method followed by mass spectrometry (FIG. 4 and FIG. 2A).

To date, it has been unclear whether MTBR tau could be involved in extracellular tau propagation because extracellular levels were thought to be too low to seed and spread the pathology. These new findings of the stoichiometry of MTBR in CSF support the hypothesis that MTBR-containing species could spread extracellularly as pathological species. These measures also inform potential targets of anti-tau drugs in development for Alzheimer's disease and provide a quantitative measure of the target as shown by the two-fold to three-fold increase of MTBR tau species in CSF from Alzheimer's disease patients. However, a limitation is that the pathological species may be present in the interstitial fluid (ISF) rather than CSF (Colin et al., 2020). Although some reports revealed that CSF tau originates mainly from ISF (Reiber, 2001) and human CSF from Alzheimer's disease patients can induce tau seeding in a transgenic mice model (Skachokova et al., 2019), further investigations are necessary to address if tau species detected in CSF reflect pathological tau which can propagate in human brain.

Previous studies show that inoculation with Alzheimer's disease brain tau aggregates into mouse brain induced severe tau pathology (Guo et al., 2016; Narasimhan et al., 2017); however, there have been no reports that identify the pathological tau species within the extracellular space that is also linked to disease progression in humans. This led to the testing of whether CSF MTBR tau species change in Alzheimer's disease, and exploration of their suitability as novel Alzheimer's disease biomarkers. It was found that CSF MTBR tau levels are elevated in Alzheimer's disease and are consistent with species enriched in Alzheimer's disease brain insoluble fractions. The finding that CSF MTBR tau correlates with Alzheimer's disease clinical stage and tau pathology suggests that MTBR tau is related to the mechanism of tau propagation in Alzheimer's disease, although the nature (i.e., monomeric, oligomeric, or fibril species) and origin of extracellular CSF MTBR tau are still unknown. It is possible that CSF MTBR tau may originate from brain aggregates or from neurons that actively secrete a monomeric species, and future studies should be designed to address this issue.

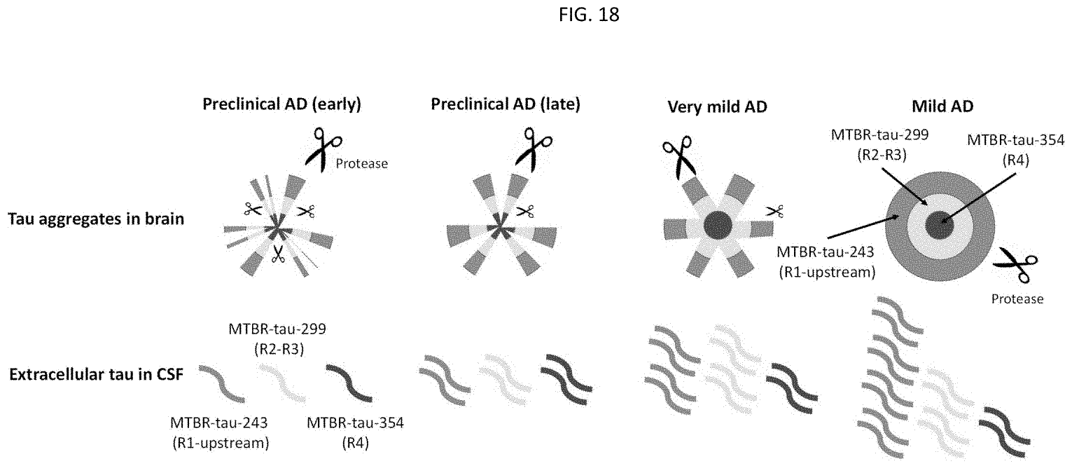

Interestingly, the trajectories of the change in CSF MTBR tau species were found to be distinct across different regions of the MTBR and at each clinical stage of Alzheimer's disease. We posit that this finding is due to structural changes in tau as determined by recent Cryo-EM findings. Cryo-EM analysis suggests the ordered β-sheet core of tau aggregates starts at residue 306 (Fitzpatrick et al., 2017). Thus, MTBR tau-354 (containing residue 354-369), MTBR tau-299 (containing residue 299-317) and MTBR tau-243 (containing residue 243-254) represent the internal side, border, and external side of the filament core, respectively. In contrast to both MTBR tau-354 and MTBR tau-299, MTBR tau-243 levels were incrementally greater across all disease stages. MTBR tau-243 and the nearby region (i.e., residue 226-230) levels in CSF are also highly correlated with tau PET SUVR performance (FIG. 7 and Table 5), which supports the hypothesis that MTBR tau-243 and potentially the nearby region deposit into brain tau aggregates and are also secreted extracellularly (FIG. 18).

The findings that MTBR tau highly correlates with Alzheimer's disease pathology and clinical progression stages provide important insights into promising targets for therapeutic anti-tau drugs to treat tauopathies. For example, a novel tau antibody, recognizing an epitope in the upstream region of MTBR (residue 235-250) demonstrated a significant and selective ability to mitigate tau seeding from Alzheimer's disease and progressive supranuclear palsy brains in cell-based assays (Courade et al., 2018). These findings suggest that the upstream region of MTBR could be related to extracellular, pathological tau. This is supported by the antibody mitigated propagation of tau pathology to distal brain regions in transgenic mice that had been injected with human Alzheimer's disease brain extracts (Albert et al., 2019). Another novel tau antibody, recognizing an epitope in the upstream region of MTBR (residue 249-258) demonstrated the reduction of inducing tau pathology in cellular- and in vivo transgenic mice models seeded by human Alzheimer's disease brain extracts (Vandermeeren et al., 2018). Antibodies targeting MTBR tau-299 and MTBR tau-354 species also mitigated tau pathology induced by seeding of P301L tau or Alzheimer's disease brain extract (Weisová et al., 2019; Roberts et al., 2020), which supports the hypothesis that species containing specific regions of MTBR are responsible for the spread of tau pathology in tauopathies.

In summary, it was discovered that MTBR tau species in CSF exist as C-terminal fragments and are specifically increased in Alzheimer's disease, reflecting the enrichment seen in Alzheimer's disease brain aggregates. The findings suggest specific MTBR-containing species (MTBR tau-299 and MTBR tau-243) are promising CSF biomarkers to measure amyloid and tau pathology in Alzheimer's disease. In particular, the mid-domain-independent MTBR tau-243 paralleled disease progression and tau pathology in Alzheimer's disease and may be utilized as a biomarker of tau pathology and a target for novel anti-tau antibody therapies.

### Example 2

Murine monoclonal antibodies that specifically bind MTBR tau will be generated, sequenced, and characterized for their binding properties.

Briefly, to generate the antibodies, 100 μg of antigen (in 200 μl PBS+200 μl complete Freund's adjuvant) is injected intraperitoneally (IP) on day 0, day 14 and day 28 into a relevant mouse strain. A last boost of 50 μg of antigen in PBS is injected IP 3 days before fusion of myeloma cells with spleen cells of the mice. Serum is tested by direct ELISA to the antigen on day 21 and day 35. If titer is over 1:10,000, myeloma cells are then fused with mouse spleen cells per standard protocol, followed by isolation of hybridoma clones. Antibodies are generated using tau knockout mice (Jackson Labs, www.jax.org/strain/00725; Bar Harbor, Maine; Dawson et al., J Cell Sci, 2001, 114:1179-1187 or wild-type mice on a B6/C3 background. The antigen is human recombinant tau peptides of at least 10 amino acids in length and comprising at least consecutive 4 amino acids of SEQ ID NO: 3, SEQ ID NO: 6 or SEQ ID NO: 7, including human recombinant tau peptides comprising SEQ ID NO: 3, SEQ ID NO: 6 or SEQ ID NO: 7.

To initially screen antibodies, supernatants from hybridoma cells are added to 96-well plates coated with the tau peptides listed above and bound antibodies are detected using anti-mouse IgG HRP. The antibodies that perform well in the initial screening will be further characterized in a tau-seeding assay as described in Holmes et al., Proc Natl Acad Sci USA, 2014, 111(41): E4376-85, and antibodies that inhibit tau seeding and spreading will be selected for further studies. For instance, specificity of binding to disease specific moieties at MTBR-243, MTBR-299, MTBR-354, and associated MTBR regions in human brain, CSF and blood will be determined to characterize which regions and tau species the antibodies bind. Anti-MTBR tau antibodies will also be evaluated by performing competition experiments with human AD pathology, including brain, CSF and blood, by combining several different antibodies in sequential immunoprecipitations with characterization and comparison between antibodies to determine the tau species, isoforms, PTMs, neo-epitopes and fragments.

Further, sequential neo-epitopes can be synthesized and testing of each antibody can occur confirm the neo-epitope antibody avoids non-specific binding to full-length tau and possibly other tau isoforms. These neo-epitopes will be identified and characterized on human AD tissue and fluids.

### Example 3

It is expected that immunization with epitope binding agents of the present disclosure will prevent tau spreading induced by extracellular pathological tau species, such as synthetic (in vitro) pre-formed tau fibrils and tau derived from tauopathy-related pathology in a human brain which has a unique tau structural component (e.g., tau partially or completely purified from human Alzheimer's disease pathology, etc.). Tau seeding and propagation can be evaluated by methods known in the art, including cell-based assays or, more preferably, in studies where pre-formed tau fibrils and/or pathological tau from human brains are intracerebrally injected into mice to observe ipsilateral and contralateral spreading of tau pathology.

For instance, four-month-old htauP301L transgenic mice can be treated with intraperitoneal injections (30 mg/kg) of isotype control antibodies or an anti-tau antibody of Example 2 7 days and 24-hours before stereotaxic injection, in the right hippocampus, of P301L-K18 fibrils. Antibodies are then administered intraperitoneally once a week. Mice are sacrificed 6-weeks post-injection plus 24 h. Alternatively, 1-month-old Tg30tau mice can be treated with intraperitoneal injections (30 mg/kg) of isotype control antibodies or an anti-tau antibody of Example 2 7 days and 24 hours before stereotaxic injection, in the right hippocampus, of extracts of human brain Alzheimer's disease pathology. Antibodies are then administered intraperitoneally once a week. Mice are sacrificed 6-weeks post-injection plus 24 h. Whole brains are processed for immunohistochemical analysis using AT8 antibody. A statistically significant decrease in AT8 immunoreactivity percent for the anti-tau antibody as compared to the isotype control indicates immunization with the anti-tau antibody prevents tau spreading induced by extracellular pathological tau species.

### Example 4

It is expected that treatment with the anti-tau antibodies will reduce tau pathology and improve symptoms associated with pathological tau deposition in a relevant animal model, such as P301S Tau Tg mice, human tau spreading models including injection of human AD tau pathology, and other models that recapitulate human tau AD pathology.

For instance, it is expected that treatment with anti-tau antibodies of Example 2 and 3 will reduce neuritic plaque (NP) tau pathology in an animal model where endogenous mouse tau is induced to have Alzheimer's disease (AD)-like tau features by seeding the brain with sarkosyl-insoluble tau aggregates isolated from the frontal cortex of human AD brain tissue (AD-tau). Experimental details are as described in Leyns et al. Nature Neuroscience, 2019, 22(8): 1217-1222 with a few exceptions. First, 5X FAD mice (www.jax.org/strain/008730) are used to inject the human AD tau not the mouse strains described in Leyns et al. Second, anti-tau antibody or control antibody are injected once weekly for 3 months at the time of AD-tau seeding. Inhibition of AD-tau spreading from the site of the seeding indicates a reduction in NP tau pathology.

Alternatively, in P301S mouse colonies, mice first develop intracellular tau pathology beginning at 5 months of age. To test the efficacy of the antibodies by chronic intracerebroventricular (ICV) administration, a catheter is surgically implanted into the left lateral ventricle of each mouse at 6 months of age and anti-tau antibodies are continuously infused for 3 months via an Alzet subcutaneous osmotic mini-pump. Anti-Aβ antibody HJ3.4 and phosphate buffered saline (PBS) are typically used as negative controls. After 6 weeks, each pump is replaced with one filled with fresh antibody solution or PBS. At the time of brain dissection (e.g., 1, 2, 3 or more months of infusion), catheter placement in the left lateral ventricle of each mouse is verified by cresyl violet staining. Only mice with correctly placed catheters are included in subsequent analyses. It is expected that anti-tau treatment will reduce tau pathology in P301S mice after treatment for 3 months or more. To determine the extent of tau pathology in P301S mice after treatment, multiple stains for tau pathology are typically carried out.

For instance, brain sections can be assessed by immunostaining with the anti-phospho tau antibody AT8. AT8 binds phosphorylated residues Ser202 and Thr205 of both mouse and human tau. For AT8 staining, 3 brain sections from each mouse separated by 300 μm can be used (e.g., corresponding approximately to sections at Bregma coordinates −1.4, −1.7, and −2.0 mm in the mouse brain atlas). The brain sections are blocked with 3% milk in Tris-buffered saline (TBS) and 0.25% (vol/vol) Triton-X followed by incubation at 4° C. overnight with the biotinylated AT8 antibody (Thermo Scientific, 1:500). These sections are used to determine the percentage of area occupied by abnormally phosphorylated biotinylated AT8 antibody staining. All converted images are uniformly thresholded to quantify AT8 staining and the average of all three sections are used to determine the percentage of area covered by abnormally phosphorylated tau staining for each mouse. In control mice (e.g., mice treated with PBS and HJ3.4), AT8 strongly stains neuronal cell bodies and the neuropil in multiple brain regions, particularly in the piriform cortex, entorhinal cortex, amygdala, and hippocampus. It is expected that treatment with anti-tau antibodies of Example 2 will reduce AT8 staining.

Biotinylated PHF1 antibody (1:200) which recognizes abnormally phosphorylated tau at residues ser396 and ser404 can also be used. For PHF-1, two brain sections from each mouse, separated by 300 μm, can be used (e.g., corresponding to bregma coordinates −2.3 and −2.6 mm in the mouse brain atlas, particularly when AT8 staining also occurs). PHF1 antibody staining is expected to correlate with AT8 staining.

Thioflavin S (ThioS), or other stains known in the art, can be used to assess tau amyloid deposition in brain sections adjacent to those used for AT8 or PHF-1 staining. To determine ThioS staining, brain sections from randomly selected mice from all the treated groups (N=6) are stained in ThioS in 50% ethanol (0.25 mg/ml) for 3 min, followed by washing in 50% ethanol and distilled water. Slices are then mounted, dried and images are assessed by microscopy with the Nanozoomer. ThioS staining can be semi-quantitatively assessed using a blinded rater who gives a score from 1 (no staining) to 5 (maximum staining) in all control and anti-tau antibody treated mice. By semi-quantitative assessment, the anti-tau antibodies are expected to reduce ThioS staining compared to control mice (e.g., PBS and HJ3.4).

The ability of the anti-tau antibody treatments to rescue cognitive deficits in P301S mice is evaluated by assessing the performance of the mice on the conditioned fear procedure. Briefly, the mice are trained and tested in two Plexiglas conditioning chambers (26 cm×18 cm, and 18 cm high) (Med-Associates, St. Albans, Vt.) with each chamber containing distinct and different visual, odor, and tactile cues. Each mouse is placed into the conditioning chamber for a 5-min trial and freezing behavior was quantified during a 2-min baseline period. Beginning at 3 min and at 60-s intervals thereafter, the mice are exposed to 3 tone-shock pairings where each pairing includes a 20-s presentation of an 80 dB tone (conditioned stimulus; CS) consisting of broadband white noise followed by a 1.0 mA continuous footshock (unconditioned stimulus; CS) presented during the last second of the tone. Broadband white noise is used instead of a frequency-specific tone in an effort to avoid possible auditory deficits that might occur with age. The mice are placed back into the conditioning chamber the following day and freezing behavior was quantified over an 8-min period to evaluate contextual fear conditioning. Twenty-four hours later, the mice are placed into the other chamber containing different cues and freezing behavior is quantified during a 2-min “altered context” baseline and over the subsequent 8 min, during which time the auditory cue (tone; CS) is presented. Freezing is quantified using FreezeFrame image analysis software (Actimetrics, Evanston, Ill.), which allows for simultaneous visualization of behavior while adjusting a “freezing threshold,” which categorizes behavior as freezing or not freezing during 0.75 s intervals. Freezing is defined as no movement except for that associated with normal respiration, and the data are presented as percent of time spent freezing. To assess the extent of contextual fear conditioning, analyses are conducted within each treatment group which involves comparing the percent time spent freezing averaged over the 2-min baseline on day 1 with the averaged percent time spent freezing during the first 2 min of the contextual fear test on day 2, as well as with freezing levels averaged across the entire 8-min session. Shock sensitivity is evaluated following completion of the conditioned fear testing, according to previously described procedures in Khuchua et al. (2003; Neuroscience 119, 101-111). On day 1, all groups of mice typically exhibit similar levels of baseline freezing during the first two minutes in the training chamber, as well as similar levels of freezing during the tone-shock (T/S) conditioned stimulus-unconditioned stimulus (CS-US) pairings. Robust differences in freezing levels from the contextual fear test conducted on day 2 between anti-tau antibody groups and the PBS+HJ3.4 control mice are expected, indicating that anti-tau antibodies preserve associative learning as demonstrated by rescuing contextual fear deficits.

### Example 5

Tau MTBR Antibodies will also be evaluated by performing competition experiments with human AD pathology, including brain, CSF and blood, by combining several different antibodies in sequential immunoprecipitations with characterization and comparison between antibodies to determine the tau species, isoforms, PTMs, neo-epitopes and fragments. Further, sequential neo-epitopes will be synthesized and testing of each antibody will occur to determine if a neo-epitope antibody can be developed to avoid non-specific binding to other tau isoforms to make a highly specific neo-epitope tau antibody which doesn't bind full length tau. These neo-epitopes will be identified and characterized on human AD tissue and fluids. This will be compared to non-AD tauopathies and normal controls.

What is claimed is:

1. An isolated antibody or antigen-binding fragment, wherein the isolated antibody or antigen-binding fragment specifically binds tau and
(a) recognizes an epitope within SEQ ID NO: 3 (LQTAPVPMPDLK), SEQ ID NO: 6 (HVPGGGSVQIVYKPVDLSK), and SEQ ID NO: 7 (IGSLDNITHVPGGGNK); or
(b) recognizes an epitope comprising four or more continuous amino acids of an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK); or
(c) recognizes an epitope comprising an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK).

2. The isolated antibody of claim 1, wherein the isolated antibody is specifically able to block tau seeding activity and/or tau spreading in an in vitro cell assay or an in vivo animal model.

3. The isolated antibody of claim 1, wherein the isolated antibody or antigen-binding fragment is an IgG1 isotype.

4. The isolated antibody of claim 1, wherein the isolated antibody or antigen-binding fragment is an IgG4 isotype.

5. The isolated antibody of claim 1, wherein the isolated antibody or antigen-binding fragment comprises a variable region, and the variable region comprises a framework region that has at least 75% sequence identity with a human framework region sequence.

6. The isolated antibody of claim 1, wherein the isolated antibody or antigen-binding fragment comprises one or more constant regions, or a portion of a constant region, that has at least 90% sequence identity with human constant region sequence.

7. The isolated antibody of claim 1, wherein the isolated antibody is a monoclonal antibody.

8. The isolated antibody of claim 1, wherein the isolated antibody antigen-binding fragment is a single chain Fv fragment (scFv), an F(ab′) fragment, an F(ab) fragment, or an F(ab′)₂ fragment.

9. An immunoassay comprising an isolated antibody or antigen-binding fragment, wherein the isolated antibody or antigen-binding fragment specifically binds tau and
(a) recognizes an epitope within SEQ ID NO: 3 (LQTAPVPMPDLK), SEQ ID NO: 6 (HVPGGGSVQIVYKPVDLSK), and SEQ ID NO: 7 (IGSLDNITHVPGGGNK); or
(b) recognizes an epitope comprising four or more continuous amino acids of an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK); or
(c) recognizes an epitope comprising an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK).

10. A kit comprising an isolated antibody or antigen-binding fragment, wherein the isolated antibody or antigen-binding fragment specifically binds tau and
(a) recognizes an epitope within SEQ ID NO: 3 (LQTAPVPMPDLK), SEQ ID NO: 6 (HVPGGGSVQIVYKPVDLSK), and SEQ ID NO: 7 (IGSLDNITHVPGGGNK); or
(b) recognizes an epitope comprising four or more continuous amino acids of an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK); or
(c) recognizes an epitope comprising an amino acid sequence selected from the group consisting of SEQ ID NO: 3 (LQTAPVPMPDLK), SEQ ID NO: 6 (HVPGGGSVQIVYKPVDLSK), and SEQ ID NO: 7 (IGSLDNITHVPGGGNK).


SEQUENCE LISTING (21 sequences)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 4 aa, Artificial Sequence
  Synthesized
IGST

SEQ ID NO: 3 - protein, 12 aa, Artificial Sequence
  Synthesized
LQTAPVPMPDLK

SEQ ID NO: 4 - protein, 4 aa, Artificial Sequence
  Synthesized
VQII

SEQ ID NO: 5 - protein, 4 aa, Artificial Sequence
  Synthesized
LDLS

SEQ ID NO: 6 - protein, 4 aa, Artificial Sequence
  Synthesized
HVPG

SEQ ID NO: 7 - protein, 4 aa, Artificial Sequence
  Synthesized
IGSL

SEQ ID NO: 8 - protein, 15 aa, Artificial Sequence
  Synthesized
IGSLDNITHVPGGGN

SEQ ID NO: 9 - protein, 4 aa, Artificial Sequence
  Synthesized
VQIV

SEQ ID NO: 10 - protein, 4 aa, Artificial Sequence
  Synthesized
TPPS

SEQ ID NO: 11 - protein, 18 aa, Artificial Sequence
  Synthesized
QEFEVMEDHAGTYGLGDR

SEQ ID NO: 12 - protein, 20 aa, Artificial Sequence
  Synthesized
DQGGYTMHQDQEGDTDAGLK

SEQ ID NO: 13 - protein, 23 aa, Artificial Sequence
  Synthesized
ESPLQTPTEDGSEEPGSETSDAK

SEQ ID NO: 14 - protein, 20 aa, Artificial Sequence
  Synthesized
STPTAEDVTAPLVDEGAPGK

SEQ ID NO: 15 - protein, 39 aa, Artificial Sequence
  Synthesized
QAAAQPHTEIPEGTTAEEAGIGDTPSLEDEAAGHVTQAR

SEQ ID NO: 16 - protein, 5 aa, Artificial Sequence
  Synthesized
IATPR

SEQ ID NO: 17 - protein, 15 aa, Artificial Sequence
  Synthesized
SGYSSPGSPGTPGSR

SEQ ID NO: 18 - protein, 10 aa, Artificial Sequence
  Synthesized
TPSLPTPPTR

SEQ ID NO: 19 - protein, 5 aa, Artificial Sequence
  Synthesized
VAVVR

SEQ ID NO: 20 - protein, 10 aa, Artificial Sequence
  Synthesized
TDHGAEIVYK

SEQ ID NO: 21 - protein, 11 aa, Artificial Sequence
  Synthesized
SPVVSGDTSPR